(19) United States
(12) Patent Application Publication
(10) Pub. No.: US 2016/0291028 A2
(43) Pub. Date: Oct. 6, 2016

(54) METHOD FOR DETECTINGAND/OR ASSAYING ANNEXIN A3 FROMA MAMMAL IN BLOOD OR AT LEAST ONE DERIVATIVE THEREOF

(71) Applicants: Yasemin ATAMAN-ONAL, Reyrieux (FR); Genevieve CHOQUET-KASTYLEVSKY, Francheville (FR); Hader HAIDOUS, Saint-Etienne (FR); Sandrine MICHEL-BUSSERET, Lyon (FR); Catherine OTT, Bron (FR)

(72) Inventors: Yasemin ATAMAN-ONAL, Reyrieux (FR); Genevieve CHOQUET-KASTYLEVSKY, Francheville (FR); Hader HAIDOUS, Saint-Etienne (FR); Sandrine MICHEL-BUSSERET, Lyon (FR); Catherine OTT, Bron (FR)

(73) Assignee: bioMerieux, Marcy lEtoile (FR)

(21) Appl. No.: 14/405,970

(22) PCT Filed: Jun. 5, 2013
(86) PCT No.: PCT/EP2013/061593

§ 371 Date: Dec. 5, 2014

(30) Foreign Application Priority Data
Jun. 7, 2012 (FR) 1255340

(51) Int. Cl. G01N 33/574

(52) U.S. Cl. CPC G01N 33/57496; G01N 2333/4718

(57) ABSTRACT

Method and pack for detecting and/or assaying annexin A3 from a mammal in blood or at least one derivative thereof.

The present invention relates to a method and a kit for detecting and/or assaying Annexin A3 from a mammal (for example a human) in the blood or in the biological fluids which derive therefrom, such as serum or plasma. A detection and/or an assaying of this kind is in fact desirable particularly for the purpose of diagnosis, prognosis or to determine the susceptibility to acute or chronic pathologies.

Annexin A3 (ANXA3) belongs to the Annexin family. The proteins of this family have the common characteristic of being able to attach to the phospholipid membranes in the presence of calcium. These proteins are expressed relatively ubiquitously in various cellular tissues and types, in animals and plants. There are 12 Annexins that have been identified in vertebrates, and these form the Annexin A family

The property characterising Annexins, namely their ability to bind the membranal phospholipids in a calcium-dependent manner, results from structural characteristics shared by all members of this family of proteins. Thus, in its protein sequence, each Annexin contains repeat domains called “Annexin repeat domains”, which are 4 or 8 in number (4 for Annexin A3), and have a length of about 70 amino acids Each repeat domain, also called endonexin domain, is folded in 5 alpha helices and contains a characteristic type 2 pattern (GxGT - - D/E) which serves to attach Ca²⁺ ions. The Annexins of the animal kingdom have variable N-terminus domains, which often feature the additional functional specificities of proteins. Crystallographic analyses show that the secondary and tertiary structures of the Annexins are preserved even if the identity of the amino acid sequences does not exceed 45 to 55%. The 4 repeat domains form a structure that resembles a disc, with a slightly convex surface on which the Ca²⁺ attachment sites are located, and a concave surface where the N- and C-terminus ends of the protein are located in proximity

Thus, owing to their structural properties, Annexins can form networks on the membrane surface and organize membrane microdomains and recruitment sites for interactive proteins. More generally, they are implicated in the plasma-cytoskeleton membrane link, the regulation of the calcium ion flows across the membranes and the exo- and endocytotic traffic Hence, Annexins play an important role in cellular differentiation, cellular migration and immunomodulation. Most Annexins are also phospholipase A2 inhibitors and have anticoagulant properties. Moreover, each member of the family may be implicated individually in other physiological or pathological processes.

Annexin A3 is expressed in various tissues such as lung, spleen, placenta, prostate, kidney It is considered to be a rare Annexin in comparison with other members of the family which have a more ubiquitous expression and/or higher levels of expression. Annexin A3 is very abundant in human neutrophils, where it accounts for about 1% of the cytosolic proteins Initially, two isoforms of Annexin A3 were described, one having an apparent molecular weight of 33 kDa, mainly present in the neutrophils, and the other having an apparent molecular weight of 36 kDa, found in the monocytes. The difference in molecular weight between the two isoforms does not result from a post-translational modification such as phosphorylation or glycosylation. In 2010, it was demonstrated that an alternative splicing phenomenon could be one mechanism explaining the presence of these two isoforms. Thus, the authors identified two complementary DNA variants of Annexin A3: the first, with a length of 973 base pairs (bp) codes for an entire protein of 323 amino acids corresponding to the 36 kDa isoform; the second, with a length of 885 bp, does not contain the exon III due to an alternative splicing. When this exon is not present, the open reading frame is interrupted, and the translation only becomes possible from the ATG codon present in the exon IV. In consequence, the first 39 amino acids of the protein are not expressed, giving rise to a truncated protein with an N-terminus end of 284 amino acids, corresponding to the 33 kDa isoform Moreover, the Western blot analysis after a 2-dimensional electrophoresis (2DE-WB) shows that the 36 kDa isoform of Annexin A3 migrates in the form of 4 to 6 spots, having different isoelectric points. The 36 kDa isoform identified by a 1-dimensional Western blot actually corresponds to a group of heterogeneous isoforms

With regard to its function, it has been demonstrated that Annexin A3 is a lipocortin, that is to say, a phospholipase A2 inhibitor Moreover, it exerts anticoagulant activity in the placenta and causes the aggregation of the membranal vesicles, such as, for example, the neutrophil granules In vitro, the presence of the Annexin A3 protein is necessary for the growth of rat hepatocytes

More generally, the Annexin A3 protein is implicated directly or indirectly in many pathologies.

In the prior art, Annexin A3 has frequently been associated with different types of cancers. For example, Patent Application EP-A-1724586 describes the determination of the abundance of intracellular and/or extracellular Annexin A3 in a sample of urine with a view to diagnosing a urogenital cancer or a cancer of the gastrointestinal tract. In particular, Annexin A3 is significantly associated with the progression and with the various stages of prostate diseases and prostate cancers. In prostate tissue, and in urine, the level of expression of the Annexin A3 protein decreases in case of prostate cancer

With regard to colorectal cancer, it is known that the expression of mRNA coding for Annexin A3 and the expression of the Annexin A3 protein are increased in colorectal tumours in comparison with the healthy adjacent tissue Moreover, Annexin A3 is used in a method for determining the probability of having a colorectal cancer in a subject (WO 2009/125303). For this purpose, whole blood is collected in PAXgene Blood RNA tubes (PreAnalytiX)™ for stabilising the intracellular RNA of the cells present in the peripheral blood, the RNA is extracted, then the level of the expression of RNA coding for Annexin A3 is measured by quantitative RT-PCR in real time. However, this level of expression of RNA coding for Annexin A3 reflects the host's immune response to the pathology. It is thus an indirect nonspecific marker of cancerous cells which necessitates the combination with other genes to improve specificity. It has thus been shown that the combination with six other genes serves to improve clinical performance Furthermore, this molecular biology technology is expensive.

With regard to lung cancer, it is known that the expression of the Annexin A3 protein is significantly high in primary adenocarcinomas having local metastases in the lymph nodes, compared with primary tumours which have not metastasised. This result suggests that Annexin A3 plays an important role in the progression of lung cancer and that it is a prognostic factor for this pathology

Annexin A3 also offers diagnostic value for pancreatic cancer

Moreover, the decrease in the expression of Annexin A3 is correlated with the tumoral progression in papillary thyroid cancer this protein also being a candidate biomarker in connection with a predisposition to breast cancer (cf. US 2011/0251082).

Furthermore, links also exist between the expression of Annexin A3 and the inflammatory process. Cyclooxygenase-2 (COX-2) is a peroxidase that serves to convert arachidonic acid to prostaglandin H2. This is an inducible enzyme which is expressed at the inflammation site and which has a pro-inflammatory action. The inhibition of COX-2 by non-steroidal anti-inflammatory medication serves to decrease the expression of inflammatory mediators and has analgesic effects. Annexin A3 forms part of the genes whose expression is increased following treatment by rofecoxib (a selective COX-2 inhibitor) and which are associated with the inhibition of phospholipase A2 and with the suppression of cytokine cascades Annexin A3 is implicated in the signalling pathways serving to control the inflammation, and is therefore useful as a marker or therapeutic target in any pathology involving acute or chronic inflammation.

The systemic inflammatory response syndrome or SIRS designates a generalised inflammatory state which can be induced by an infection or a non-infectious cause such as, for example, a traumatism, a serious burn, or even a pancreatitis. More precisely, a SIRS resulting from a documented infection, that is to say, confirmed in the laboratory, is called sepsis. The microbiological tests are decisive: they serve to identify the infectious agent causing the infection and, if necessary, to test its antibiotic resistance profile. Haemoculture is a key examination that must be conducted in optimal conditions in order to obtain the maximum chance of detecting the microorganism responsible for the infection as quickly as possible. However, the responsible infectious agent (bacteria, virus, fungus or parasite) is only statistically identified in about 2 cases out of 3. It is thus necessary to take into consideration other signs and symptoms, as well as the results of clinical and biological examinations, in order to be able to pose the diagnosis, determine the prognosis and monitor the response to treatment. A biomarker, procalcitonin (PCT) serves to improve the clinical performances for the early diagnosis of sepsis and the choice of an appropriate treatment. The expression of PCT is highly specific for the bacterial origin of the systemic inflammation syndrome and it also occurs very early: an increase in the serous rate is measurable in 3 to 6 h after the start of the infection. When the infectious agent is non-bacterial, the diagnosis of sepsis is more difficult to pose. More generally, it is very useful to be able to predict the development of sepsis in patients exhibiting a SIRS state. For this purpose, Patent Application US 2011/0105350 describes methods using the combination of mRNA markers measured in the RNA extracts obtained from blood samples. Annexin A3 forms part of these chosen markers.

Patent Application WO 2010/011860, for its part, discloses methods for detecting pre-diabetes and diabetes. These methods are based on the comparison of proteomic profiles which contain at least one unique expression signature that is characteristic of pre-diabetes or diabetes. These proteomic profiles contain data on the expression of various proteins, including Annexin A3.

Although Annexin A3 is recognised as a marker for cancer and other pathologies, the methods for detecting it and quantifying it described so far either lack specificity in the sense that the antibodies used undergo cross-reactions with other Annexins (cf. WO 2006/125580), or lack sensitivity for its detection in a complex biological fluid such as urine (cf. WO 2007/141043). In general, the prior art methods lack robustness and do not allow the detection and reliable quantification of very low Annexin A3 concentrations of the order of ng/mL in a complex biological fluid and, in particular, in the blood, or at least one of its derivatives such as plasma and serum.

Up to now, no immunodetecting and/or immunoassaying technique would make it possible to detect and/or assay Annexin A3 in the blood, plasma or serum (particularly in quantities of the order of ng/mL). Such blood, plasma or serum assaying is extremely desirable in the sense that it is easy to carry out (by simply taking blood), easily reproducible and the result obtained is not based in whole or in part on the success of an earlier manipulation of the patient such as a digital prostate examination aiming to recover an expressed urine sample (in the case of suspected prostate cancer).

Unexpectedly, the inventors have discovered that in order to overcome all or some of the abovementioned disadvantages, it would be advisable to use a pair of antibodies, one of which is directed against the first repeat domain (D1) of Annexin A3 from a mammal and the other is directed against the fourth repeat domain (D4) of Annexin A3 from said mammal. If human Annexin A3 is considered, a pair of antibodies should be used, one of which is directed against the first repeat domain of human Annexin A3 whose sequence is identified as SEQ ID NO: 1 (amino acids 27-87 of human Annexin A3, according to the numbering of the UniProtKB database of human complete ANXA3 accession No. P12429) and the other is directed against the fourth repeat domain of human Annexin A3 whose sequence is identified as SEQ ID NO: 2 (amino acids 258-318, according to the numbering of the UniProtKB database of human complete ANXA3 accession No. P12429).

In the description below, the sequence of the first repeat domain of human Annexin A3 is identified as SEQ ID NO: 1, the sequence of the fourth repeat domain of the human Annexin A3 is identified as SEQ ID NO: 2, the sequence of the second repeat domain of human Annexin A3 is identified as SEQ ID NO: 3, the sequence of the third repeat domain of human Annexin A3 is identified as SEQ ID NO: 4. The sequence identified as SEQ ID NO: 5 corresponds to the amino acid sequence of complete human Annexin A3 (UniProtKB accession No. P12429).

“Detecting the presence of Annexin A3 in the blood or at least one of its derivatives” (biological fluids such as plasma, serum, etc.) is understood to mean revealing the presence of this protein, in particular in the abovementioned biological fluids.

“Assaying (quantifying) Annexin A3 in the blood or at least one of its derivatives” (biological fluids such as plasma, serum, etc.) is understood to mean determining the quantity (for example the concentration) of Annexin A3 in particular within said biological fluids.

Consequently, the aim of the present invention is a method of detecting and/or assaying Annexin A3 from a mammal in the blood or at least one of its derivatives such as plasma and serum, wherein the blood or at least one of its derivatives is brought into contact with a first antibody and a second antibody, the first antibody being directed against the first repeat domain of Annexin A3 from said mammal and the second antibody being directed against the fourth repeat domain of Annexin A3 from said mammal. If human Annexin A3 is considered, the first antibody is directed against the first repeat domain of human Annexin A3, the sequence of which is identified as SEQ ID NO: 1, and the second antibody is directed against the fourth repeat domain of human Annexin A3, the sequence of which is identified as SEQ ID NO: 2.

In other words, the first antibody recognises and binds/attaches to all or part of the first repeat domain of Annexin A3 from the mammal under investigation and the second antibody recognises and binds/attaches to all or part of the fourth repeat domain of Annexin A3 from said mammal.

This specific combination of a first antibody directed against the first repeat domain of Annexin A3 from a mammal and a second antibody directed against the fourth repeat domain of Annexin A3 from this mammal makes it possible to detect and/or assay Annexin A3 in blood, plasma or serum (particularly in quantities of the order of ng/mL) in said mammal. This is all the more unexpected because when, instead of said second antibody, an antibody directed against a repeat domain of Annexin A3 other than its fourth repeat domain (for example directed against the first repeat domain) is used, it is not possible to assay, or even in certain cases to detect Annexin A3 in the blood, plasma or serum of the mammal in question.

In human beings, the detection and/or assaying method according to the invention makes it possible to detect and/or assay the isoform of 33 kDa of human Annexin A3 and/or that of 36 kDa (actually corresponding to a group of heterogeneous isoforms see above). The isoform of 33 kDa is a truncated molecule by 39 amino acids at the N-terminus side relative to the isoform of 36 kDa. In other words, its polypeptide sequence corresponds to the amino acids 40-323 of SEQ ID NO: 5. Thus, if the first antibody chosen binds/attaches to fragment 27-39 of the first repeat domain of human Annexin A3, the assay method detects and/or assays solely the isoform of 36 kDa. In contrast, if the first selected antibody binds/attaches to fragment 40-87 of the first repeat domain of human Annexin A3, the assay method detects and/or assays all of the isoforms.

Domains D1 and D4 of Annexin A3 present a strong identity in mammals and notably in the mammals currently being studied. As a result, the method and the kit of the present invention are applied to the detection and/or assaying of Annexin A3 in the blood or one of its derivatives, such as plasma and serum, in a mammal.

In particular, the method and the kit of the present invention apply to the detection and/or assaying of Annexin A3 notably from the following species: Homo sapiens (Humans), Sus scrofa domesticus (Pig), Bos taurus (Bull), Canis familiaris (Dog), Oryctolagus cuniculus (Rabbit), Macaca Mulatta (Rhesus macaque), Gorilla gorilla gorilla (Gorilla). Advantageously the method and the kit of the present invention will very obviously be able to be implemented in human beings but also in so-called domestic mammals (such as cat, dog, etc.), as well as in primates.

Particularly interestingly, the detection and/or assaying method according to the invention is implemented in vitro on biological samples of human origin.

More precisely, to analyse the identity of protein sequences D1 and D4 of Annexin A3 in different mammals, the amino acid sequences of the first and fourth repeat domains (respectively designated “D1” and “D4”) of human Annexin A3 have been compared to those of the first and fourth repeat domains of Annexin A3 in other mammals. With this aim, the UniprotKB database (www.uniprot.org) was consulted. Table 1 hereafter shows, for each mammal species included in the present analysis, the accession number of the Annexin A3 protein sequence of the species, as well as the name of the entry within the database. For each mammal species, the sequences of domains D1 and D4 have been extracted manually and then aligned with the corresponding sequence of human Annexin A3 using the “Align” function which is available on the “Align” tab on the Uniprot internet site. This function makes use of the Clustal 0 (v1.0.3) program. Said Table 1 shows for each of the domains D1 and D4, the identity percentage relative to the corresponding sequence of human Annexin A3. The alignments of sequences of the domains D1 and D4 are respectively presented in FIGS. 1a and 1b.

TABLE 1

| | | | Identity | Identity |
|---|---|---|---|---|
| Uniprot | | | percentage | percentage |
| accession No. | Name of the entry | Organism | (D1) | (D4) |
| P12429 | ANXA3_HUMAN | Homo sapiens (Human) | 100% | 100% |
| O35639 | ANXA3_MOUSE | Mus musculus (Mouse) | 87% | 89% |
| P14669 | ANXA3_RAT | Rattus norvegicus (Rat) | 82% | 87% |
| Q3SWX7 | ANXA3_BOVIN | Bos taurus (Bull) | 87% | 92% |
| E2R0N3 | E2R0N3_CANFA | Canis familiaris (Dog) | 87% | 93% |
| G1TEM7 | G1TEM7_RABIT | Oryctolagus cuniculus | 93% | 90% |
| | | (Rabbit) | | |
| F6Z8Y2 | F6Z8Y2_MACMU | Macaca Mulatta (Rhesus | 98% | 97% |
| | | macaque) | | |
| G3R3Y9 | G3R3Y9_GORGO | Gorilla gorilla gorilla | 100% | 100% |
| | | (Gorilla) | | |

As indicated previously, the method of assaying according to the present invention can be achieved in a sample of blood or one of its derived fluids (plasma, serum, etc.). The definitions of the terms “plasma” and “serum” are the usual definitions used in biology, namely:
plasma consists of the liquid part of the blood in which the cell elements (red globules, white globules, platelets) are suspended; and
serum designates the liquid which separates from clots after coagulation of the blood, corresponding to plasma without fibrinogen.

The object of the invention is also a method of in vitro detection and/or assaying of Annexin A3 in a biological sample from a mammal, said biological sample being chosen from amongst blood or at least one of its derivatives such as plasma and serum, wherein the biological sample is brought into contact with a first antibody and a second antibody, the first antibody being directed against the first repeat domain of Annexin A3 from said mammal and the second antibody being directed against the fourth repeat domain of Annexin A3 from said mammal.

Advantageously, the first antibody is a capture antibody and the second antibody is a detection antibody (visualisation).

According to a preferred embodiment, the first antibody is selected from amongst the antibodies directed against an epitope, of which the amino acid sequence comprises at least 7 consecutive amino acids and no more than 17 consecutive amino acids of the first repeat domain (D1) of Annexin A3 from the mammal under investigation. In the case of human Annexin A3 assaying, the first antibody is selected from amongst the antibodies directed against an epitope of which the amino acid sequence comprises at least 7 consecutive amino acids and no more than 17 consecutive amino acids of SEQ ID NO: 1.

The first antibody is preferably selected from amongst the antibodies directed against an epitope included in the first repeat domain of Annexin A3 from said mammal, said epitope having an amino acid sequence selected from amongst the following sequences:
Xaa1-Xaa2-A-Q-Xaa3-Xaa4-L-I-Xaa5-Xaa6-Xaa7-Y-Q-Xaa8-Xaa9-Xaa10-Xaa11 (SEQ ID NO: 37),
L-I-Xaa5-Xaa6-Xaa7-Y-Q-Xaa8-Xaa9-Xaa10-Xaa11 (SEQ ID NO: 38),
I-Xaa5-Xaa6-Xaa7-Y-Q-Xaa8-Xaa9-Xaa10-Xaa11-Xaa12-Xaa13 (SEQ ID NO: 39),
Xaa6-Xaa7-Y-Q-Xaa8-Xaa9-Xaa10-Xaa11 (SEQ ID NO: 40),
Xaa6-Xaa7-Y-Q-Xaa8-Xaa9-Xaa10-Xaa11-Xaa12-Xaa13-L-K-Xaa14-D-L-K-G (SEQ ID NO: 41), provided that the amino acid sequence SEQ ID NO: 41 is fused on the N-terminus side to a sequence of at least 30 amino acids,
DLSGHF-Xaa20-Xaa21-Xaa22 (SEQ ID NO: 42),
LSGHF-Xaa20-Xaa21 (SEQ ID NO: 43),
wherein:
   Xaa1 represents a serine residue, a threonine residue, or an analogous amino acid,
   Xaa2 represents an asparagine residue, a serine residue or an analogous amino acid,
   Xaa3 represents an arginine residue, a histidine residue or an analogous amino acid,
   Xaa4 represents a glutamine residue, a leucine residue or an analogous amino acid,
   Xaa5 represents a valine residue, an alanine residue or an analogous amino acid,
   Xaa6 represents a lysine residue, an arginine residue or an analogous amino acid,
   Xaa7 represents a glutamic acid residue, a glutamine residue or an analogous amino acid,
   Xaa8 represents an alanine residue, a glutamic acid residue or an analogous amino acid,
   Xaa9 represents an alanine residue, a leucine residue or an analogous amino acid,
   Xaa10 represents a tyrosine residue, a cysteine residue or an analogous amino acid,
   Xaa11 represents a glycine residue, a glutamic acid residue or an analogous amino acid,
   Xaa12 represents a lysine residue, a glutamine residue or an analogous amino acid,
   Xaa13 represents a glutamic acid residue, an alanine residue or an analogous amino acid,
   Xaa14 represents an aspartic acid residue, an alanine residue or an analogous amino acid,
   Xaa20 represents a glutamic acid residue, a lysine residue or an analogous amino acid,
   Xaa21 represents a histidine residue, a glutamine residue or an analogous amino acid,
   Xaa22 represents a leucine residue, a valine residue or an analogous amino acid.

### Advantageously:

Xaa1 represents a serine residue or a threonine residue,
Xaa2 represents an asparagine residue or a serine residue,
Xaa3 represents an arginine residue or a histidine residue,
Xaa4 represents a glutamine residue or a leucine residue,
Xaa5 represents a valine residue or an alanine residue,
Xaa6 represents a lysine residue or an arginine residue,
Xaa7 represents a glutamic acid residue or a glutamine residue,
Xaa8 represents an alanine residue or a glutamic acid residue,
Xaa9 represents an alanine residue or a leucine residue,
Xaa10 represents a tyrosine residue or a cysteine residue,
Xaa11 represents a glycine residue or a glutamic acid residue,
Xaa12 represents a lysine residue or a glutamine residue,
Xaa13 represents a glutamic acid residue or an alanine residue,
Xaa14 represents an aspartic acid residue or an alanine residue,
Xaa20 represents a glutamic acid residue or a lysine residue,
Xaa21 represents a histidine residue or a glutamine residue,
Xaa22 represents a leucine residue or a valine residue.

The expression “analogous amino acid” refers to an amino acid which, when it replaces the amino acid present in the epitope under investigation, does not bring about any substantial modification of the antigenic reactivity of said epitope relative to the antibody under investigation.

The particularly preferred analogues include those which display naturally conservative substitutions, i.e. substitutions which take place in a family of amino acids. There are several family classifications of amino acids, as is known to the person skilled in the art. Thus, according to one classification example, the amino acids can be divided into four families, namely (1) the acidic amino acids such as aspartic acid or glutamic acid, (2) the basic amino acids such as lysine, arginine and histidine, (3) non-polar amino acids such as leucine, isoleucine and methionine and (4) the non-charged polar amino acids such as glycine, asparagine, glutamine, serine, threonine and tyrosine. Phenylalanine, tryptophan and tyrosine are sometimes classed as aromatic amino acids. For example, it is possible to reasonably predict that an isolated replacement of a leucine residue by an isoleucine or valine residue, of an aspartate residue by a glutamate residue, a threonine residue by a serine residue, or a similar conservative replacement of one amino acid by another amino acid which has a structural relationship, will have no major effect on the biological activity. Another example of a method which makes it possible to predict the effect of an amino acid substitution on biological activity has been described by Ng and Henikoff, 2001

In particular, in the case of the detection and/or assaying of human Annexin A3, the first antibody is selected from amongst the antibodies directed against an epitope included in SEQ ID NO: 1, said epitope having an amino acid sequence selected from amongst the following sequences:
SNAQRQLIVKEYQAAYG (SEQ ID NO: 10),
LIVKEYQAAYG (SEQ ID NO: 11),
IVKEYQAAYGKE (SEQ ID NO: 12),
KEYQAAYG (SEQ ID NO: 13),
DLSGHFEHL (SEQ ID NO: 14),
LSGHFEH (SEQ ID NO: 15),
and
KEYQAAYGKELKDDLKG (SEQ ID NO: 22), provided that the amino acid sequence SEQ ID NO: 22 is fused on the N-terminus side to a sequence of at least 30 amino acids.

If it is desired to detect and/or assay Annexin A3 in a mammal other than a human being—for example one of the mammals listed in Table 1 above—the first antibody can be selected from amongst the antibodies directed against an epitope of which the amino acid sequence corresponds to one of sequences SEQ ID NO: 10, 11, 12, 13, 14, 15 and 22 in the mammal under investigation. By way of example, within the framework of the detection and/or assaying of Annexin A3 from a dog (Canis familiaris), said first antibody is directed against an epitope included in the first repeat domain of canine Annexin A3, said epitope having an amino acid sequence selected from amongst the following sequences:
TNAQHQLIVREYQAAYG (SEQ ID NO: 44),
LIVREYQAAYG (SEQ ID NO: 45),
IVREYQAAYGKE (SEQ ID NO: 46),
REYQAAYG (SEQ ID NO: 47),
DLSGHFKQL (SEQ ID NO: 48),
LSGHFKQ (SEQ ID NO: 49), and
REYQAAYGKELKDDLKG (SEQ ID NO: 50), provided that the amino acid sequence SEQ ID NO: 50 is fused on the N-terminus side to a sequence of at least 30 amino acids.

With regard to the above-mentioned second antibody, this latter is, preferably, selected from amongst the antibodies directed against an epitope, of which the amino acid sequence comprises at least 7 consecutive amino acids and no more than 50 consecutive amino acids of the fourth repeat domain of Annexin A3 from said mammal. If the mammal in question is a human being, the second antibody is preferably selected from amongst the antibodies directed against an epitope of which the amino acid sequence comprises at least 7 consecutive amino acids and no more than 7 consecutive amino acids of SEQ ID NO: 2.

Advantageously, the second antibody is selected from amongst the antibodies directed against an epitope, of which the amino acid sequence corresponds to the amino acid sequence starting at residue 3 and ending at residue 49 of the fourth repeat domain of Annexin A3 from the mammal under investigation. Here too, if the mammal is human, the second antibody is selected from amongst the antibodies directed against an epitope of which the amino acid sequence corresponds to the amino acid sequence starting at residue 3 and ending at residue 49 of SEQ ID NO: 2.

According to a preferred embodiment of the present invention, the second antibody recognises an epitope comprising a lysine residue at position 6 of the fourth repeat domain of Annexin A3 from a mammal and/or an aspartic acid residue at position 49 of said fourth repeat domain of Annexin A3 from said mammal. Preferably, said epitope comprises a lysine residue at position 6 of the fourth repeat domain of Annexin A3 from a mammal and an aspartic acid residue at position 49 of said fourth repeat domain of Annexin A3 from said mammal.

Within the framework of a method for detecting and/or assaying human Annexin A3, the second antibody recognises an epitope comprising a lysine residue at position 6 of SEQ ID NO: 2 and/or an aspartic acid residue at position 49 of SEQ ID NO: 2. Advantageously, the epitope recognised by this second antibody comprises both a lysine residue at position 6 of SEQ ID NO: 2 and an aspartic acid residue at position 49 of SEQ ID NO: 2. In effect, the lysine at position 6 of SEQ ID NO: 2 and the aspartic acid at position 49 of SEQ ID NO: 2 are revealed to be—during “mapping” processes—the most important residues in the antibody-antigen interaction between the second antibody and the fourth repeat domain of human Annexin A3 of sequence SEQ ID NO: 2 (cf. Example 7, section “Precise localisation of the epitope of the 13A12G4H2 and 1F10A6 antibodies”).

According to a particularly preferred embodiment, the two antibodies recognise and bind to an epitope which comprises, in addition to the lysine residue at position 6 and/or aspartic acid residue at position 49 of D4, an arginine or glutamine residue at position 3 of the fourth repeat domain of Annexin A3 from said mammal and an isoleucine or alanine residue at position 8 of said fourth repeat domain of Annexin A3 from said mammal. According to this preferred embodiment, when the detection and/or the assaying of human Annexin A3 is considered, the second antibody is directed against an epitope which comprises, in addition to the lysine residue at position 6 and/or aspartic acid residue at position 49 of SEQ ID NO: 2, the arginine residue at position 3 and isoleucine residue at position 8 of SEQ ID NO: 2. In effect, the presence of these specific amino acids at positions 3, 6, 8 and 49 of SEQ ID NO: 2 (respectively corresponding to positions 260, 263, 265 and 306 of human Annexin A3 of sequence SEQ ID NO: 5) would appear to be primordial relative to the fixing ability of the antibodies recognising the fourth repeat domain of human Annexin A3 (SEQ ID NO: 2), namely the antibodies 13A12G4H2 and 1F10A6, such as indicated within Example 7 below.

Preferably, the epitope recognised by the second antibody comprises a glycine residue at position 7 of the fourth repeat domain of Annexin A3 from said mammal, an isoleucine or alanine residue at position 8 of the fourth repeat domain of Annexin A3 from said mammal and a glycine residue at position 9 of said fourth repeat domain of Annexin A3 from said mammal. If human Annexin A3 is considered, the epitope recognised by the second antibody comprises, preferably, a glycine residue at position 7 of SEQ ID NO: 2, an isoleucine residue at position 8 of SEQ ID NO: 2 and a glycine residue at position 9 of SEQ ID NO: 2.

According to a particularly preferred embodiment, and within the framework of detecting and/or assaying human Annexin A3, the second antibody recognises an epitope which comprises an arginine residue at position 3 of SEQ ID NO: 2, a lysine residue at position 6 of SEQ ID NO: 2, a glycine residue at position 7 of SEQ ID NO: 2, an isoleucine residue at position 8 of SEQ ID NO: 2, a glycine residue at position 9 of SEQ ID NO: 2, and also an aspartic acid residue at position 49 of SEQ ID NO: 2.

According to a particularly preferred embodiment, the mammal is human and the first antibody is directed against the first repeat domain of said human Annexin A3, the sequence of which is identified as SEQ ID NO: 1, and the second antibody is directed against the fourth repeat domain of human Annexin A3, the sequence of which is identified as SEQ ID NO: 2.

The antibodies specific to the first repeat domain of human Annexin A3 (SEQ ID NO: 1) and those specific to the fourth repeat domain of human Annexin A3 (SEQ ID NO: 2), are antibodies which have a high affinity with an affinity constant of at least 10⁻⁹, preferably at least 10⁻¹⁰ and which, in addition, have a low dissociation constant lower than 2 10⁻³ s⁻¹, preferably even lower than 5 10⁻⁴ s⁻¹.

Preferably, said first antibody and/or said second antibody is/are monoclonal antibodies. Advantageously, both the first antibody and the second antibody are monoclonal antibodies. With regard to the detection and/or assaying of human Annexin A3, the preferred antibodies are the following antibodies: TGC42, TGC43 and TGC44 used as first antibodies (capture antibodies according to a preferred embodiment) and 13A12G4H2 and 1F10A6 used as second antibodies (detection antibodies according to said preferred embodiment). The preferable pair is constituted by the first antibody (capture antibody according to a preferred embodiment) TGC43 or TGC44 and the second antibody (detection antibody according to said preferred embodiment) 13A12G4H2.

The invention also relates to a method according to the present invention for the in vitro diagnosis and/or the in vitro monitoring of the evolution of a pathology comprised in the group constituted by:
cancers, such as urogenital cancers and particularly prostate cancer, colorectal cancer, lung cancer, pancreatic cancer, papillary thyroid cancer, breast cancer,
inflammatory disorders such as systemic inflammatory response syndrome, sepsis, rheumatoid polyarthritis, Crohn's disease, diabetes.

The object of the invention is also a kit for the immunodetection and/or immunoassaying of a biological sample from a mammal, said biological sample being chosen from amongst blood or at least one of its derivatives such as plasma and serum, said kit comprising a first antibody and a second antibody, the first antibody being directed against the first repeat domain of Annexin A3 from said mammal and the second antibody being directed against the fourth domain of Annexin A3 from said mammal. Said kit makes it possible to implement the method of detecting and/or assaying Annexin A3 from a mammal according to the present invention. Said first and second antibodies are as defined previously. Preferably, said blood, plasma or serum immunodetection and/or immunoassay kit makes it possible to detect and/or assay human Annexin A3. In this case, the first antibody is directed against the first repeat domain of human Annexin A3 with sequence SEQ ID NO: 1, and the second antibody is directed against the fourth domain of human Annexin A3, the sequence of which is identified as SEQ ID NO: 2.

According to a preferred embodiment, the invention relates to a kit for the immunodetection and/or immunoassaying of a biological sample from a mammal, said biological sample being chosen from amongst blood or at least one of its derivatives such as plasma and serum, said kit comprising:
a first antibody directed against the first repeat domain of Annexin A3 from said mammal, as defined in the present invention;
a second antibody directed against the fourth repeat domain of Annexin A3 from said mammal, as defined in the present invention,
   said kit making it possible to implement the method according to the present invention.

The first and second antibodies—respectively a capture antibody and a detection antibody according to a preferred embodiment—correspond to the same definitions and have the same characteristics as those presented above in relation to the detection and/or assaying method according to the present invention.

Advantageously, said kit comprises an appropriate calibration means.

“Appropriate calibration means” in the terms of the invention is understood to mean a calibration means suitable for the detection and/or assaying of Annexin A3 in the blood or one of its derivatives such as plasma and serum.

According to a preferred embodiment, the calibration means comprises a sample of blood, plasma or serum from the same origin (the same mammal). The concentration of Annexin A3 within this sample is known. Advantageously, this blood, plasma or serum of the same origin is diluted in a diluent so as to establish a standard range. Preferably, the appropriate diluent is an appropriate buffer.

By way of example, the aforesaid calibration means comprises:

Annexin A3 of endogenous origin (purified or non-purified) diluted in an appropriate buffer, or

Annexin A3 of exogenous origin (for example recombinant Annexin A3) diluted in an appropriate buffer.

In particular, the following can be cited as appropriate buffers:

Tris 0.1 M, NaCl 10-200 mM, BSA 1-20%,

Tris 0.1 M, NaCl 10-200 mM, calf serum 1-20%,

Tris 0.1 M, NaCl 10-200 mM, horse serum 1-20%,

Potassium phosphate 20-100 mM, NaCl 10-200 mM, BSA 1-20%,

Glycine 5-200 mM, HEPES 5-200 mM, BSA 1-20%.

Quite evidently, the person skilled in the art will easily determine, from his/her general knowledge, the appropriate buffers which allow an appropriate calibration means according to the present invention to be obtained.

In particular, the buffer may or may not comprise a detergent or detergents.

In addition, in order to improve the performance of the kit, additional compounds can be added, such as:
sugars: fructose, lactose, saccharose, maltose, trehalose, etc.;
anti-oxidants: ascorbic acid, methionine, etc.;
anti-reducing agents;
anti-chelating agents: EDTA, etc.;
anti-protease: aprotinin, etc.;
water-trapping polymers: polyvinylpyrolidone, dextran, etc.;
stabilisers: citric acid, etc.;
antibiotics, antimycotics, bacteriostats: sodium azide, etc.

As mentioned previously, it is essential that the calibration means be suitable for detection and/or assaying in the chosen biological sample, namely blood or at least one of its derivatives such as plasma and serum. Thus, a calibration means utilised within the framework of the detection and/or assaying of human Annexin A3 assay in expressed urine is not suitable for the blood, plasma, or serum immunodetection and/or immunoassay kit according to the invention.

Another object of the invention concerns the use of an immunodetection and/or immunoassay kit according to the present invention to implement the abovementioned detection and/or assay method.

The method of detecting and/or assaying Annexin A3 from a mammal (preferably a human being) and the immunodetection and/or immunoassay kit according to the present invention can both be utilised in an effective manner to carry out the in vitro diagnosis and/or the in vitro monitoring of the evolution of various pathologies and complaints. Amongst the pathologies thus able to be diagnosed and/or monitored are: cancers and in particular urogenital cancers (particularly prostate cancers), colorectal cancer, lung cancer, pancreatic cancer, papillary thyroid cancer, breast cancer, etc. Inflammatory pathologies can also be diagnosed/monitored. As inflammatory pathologies, it is possible to cite systemic inflammatory response syndrome (SIRS), rheumatoid polyarthritis, Crohn's disease, diabetes, etc. This list is not exhaustive.

Antibody means a polyclonal antibody, a monoclonal antibody, a humanised antibody, a human antibody or a fragment of said antibodies, in particular the fragments Fab, Fab′, F(ab′)2, ScFv, Fv, Fd. The requisite condition is that said antibodies must be specific to Annexin A3, that is to say, that they do not exhibit cross-reactions with other Annexins and they are specific to the first repeat domain of Annexin A3 or specific to the fourth repeat domain of Annexin A3; the antibodies having the highest affinities and the lowest dissociation constants are the preferred antibodies.

The polyclonal antibodies may be obtained by immunisation of an animal with the appropriate immunogen, followed by the recovery of the desired antibodies in purified form, by sampling of the serum of said animal, and separation of said antibodies from the other components of the serum, in particular by affinity chromatography on a column on which an antigen specifically recognised by the antibodies is fixed.

The monoclonal antibodies can be obtained by the hybridoma technique, of which the general principle is described below.

In a first step, an animal, generally a mouse, is immunised with the appropriate immunogen, whose B lymphocytes are then capable of producing antibodies against this antigen. These antibody-producing lymphocytes are then fused with “immortal” myelomatous cells (murines in the example) to give rise to hybridomas. Using the heterogeneous mixture of the cells thus obtained, a selection is made of the cells capable of producing a particular antibody and of multiplying indefinitely. Each hybridoma is multiplied in the form of a clone, each one leading to the production of a monoclonal antibody whose recognition properties with regard to the protein can be tested for example by ELISA, by immunotransfer (Western blot) in one or two dimensions, by immunofluorescence, or using a biosensor. The monoclonal antibodies thus selected are then purified, in particular by the affinity chromatography technique described above.

The monoclonal antibodies may also be recombinant antibodies obtained by genetic engineering, using techniques well known to a person skilled in the art.

The capture antibody is preferably fixed, directly or indirectly, to a solid support, for example a cone, a well of a microtitration plate, etc.

The detection antibody is marked using a marking reagent capable of directly or indirectly generating a detectable signal. A non-limiting list of these marking reagents includes the following:
enzymes which produce a detectable signal for example by colorimetry, fluorescence, luminescence, such as horseradish peroxidase, alkaline phosphatase, β-galactosidase, glucose-6-phosphate dehydrogenase,
chromophores such as fluorescent, luminescent, dye compounds,
fluorescent molecules such as Alexa or phycocyanines,
radioactive molecules such as ³²P, ³⁵S or ¹²⁵I.

Indirect detection systems can also be used, such as for example ligands capable of reacting with an anti-ligand. The ligand/anti-ligand pairs are well known to a person skilled in the art, which is the case for example of the following pairs: biotin/streptavidin, haptene/antibody, antigen/antibody, peptide/antibody, sugar/lectin. In this case, it is the ligand that carries the binding partner. The anti-ligand may be directly detectable by the marking reagents described in the previous section, or may itself be detectable by a ligand/anti-ligand.

These indirect detection systems may, under certain conditions, lead to a signal amplification. This signal amplification technique is well known to a person skilled in the art, and reference can be made to the prior patent applications FR98/10084 or WO-A-95/08000 to the Applicant.

Depending on the type of marking used, a person skilled in the art will add reagents for visualising the marking.

According to a preferred embodiment, the inventive method is a “sandwich” type immunoassay conducted on a blood sample or in biological fluids derived therefrom, such as serum or plasma, with or without sample treatment.

The invention will be better understood from the following examples, which are provided for illustration and which are non-limiting, and also with reference to the appended figures.

### FIGURES

FIGS. 1a and 1b respectively show the alignments of the protein sequences of the first and fourth repeat domains of Annexin A3 in eight reference mammals (including humans). In the low consensus line of each alignment, the asterisk (*) indicates a position which corresponds to a fully conserved residue; the colon (:) indicates a very conserved position, with residues which have a strong similarity (=score >0.5 in the Gonnet PAM 250 matrix); the full stop (.) indicates a moderately conserved position, with residues which possess a moderate degree of similarity (=score <0.5 in the Gonnet PAM 250 matrix). Said matrix by Gonnet et al. to which reference was made previously is described in the following publication: Gonnet G. H., Cohen M. A., Benner S. A.; “Exhaustive matching of the entire protein sequence database.”; Science 256:1443-1445 (1992)

FIG. 2 shows the calibration curves of the assays ELISA TGC44/13A12G4H2 (group 1) and TGC44/5C5B10 (group 2). For each assay, the calibration curve was plotted by assaying a concentration range of the purified native human Annexin A3 protein. The calibration curve was plotted with the human Annexin A3 concentration on the x-axis and the signal measured by the VIDAS® in RFV on the y-axis.

FIG. 3 shows the reactivity of the 4 repeat domains D1 to D4 of human Annexin A3, expressed in a recombinant form, with the 6 anti-Annexin A3 monoclonal antibodies indicated and analysed by the Western blot technique. For the TGC42 and 1F10A6 gels, the first well corresponds to the molecular weight marker.

FIG. 4 is an alignment of the sequences of the various recombinant proteins expressing human Annexin A3 domains and summarises the immunoreactivity of each of these recombinants with the TGC44 monoclonal antibody, analysed by the Western blot technique. The three dots at the N-terminus or C-terminus end of a sequence indicate that it continues in the structure, even if it is not entirely shown in the figure. The stop codon is indicated by a star (*).

FIG. 5 shows the impact of the alanine mutations of domain D4 on the recognition of human Annexin 3 by the 13A12G4H2 and 1F10A6 antibodies. The analysis was made by the ELISA technique and the capture antibody is TGC44. The tested 13A12G4H2, 1F10A6 antibodies were used for detection. The position of the alanine mutations on the protein sequence of human Annexin A3 is shown on the x-axis. The y-axis is the signal fold change which corresponds to the base 2 logarithm (mutated protein signal/non-mutated protein signal). The 5C5B10 antibody, which is not directed against domain D4, is not impacted, and serves for control. The arrows indicate the mutations for which the recognition signal was disturbed for 13A12G4H2 and 1F10A6, which serve to define the residues implicated in the fixing of these antibodies.

FIG. 6a corresponds to the immunovisualisation of the ALAscan membrane of the peptide KGDLSGHFEHLM (residues 73 to 84) of Annexin A3 comprising the epitope recognised by the antibody 5C5B10 with this antibody. The black arrow indicates a total loss of the signal; and the grey arrows indicate a significant decrease in the signal.

FIG. 6b corresponds to the immunovisualisation of the ALAscan membrane of the peptide VKEYQAAYGKEL (residues 57-68) of Annexin A3 comprising the epitope recognised by the antibody TGC43 with this antibody. The black arrow indicates a total loss or a very significant decrease in the signal.

FIG. 7 shows the absence of cross-reactivity of the ELISA assay formats described with 7 other proteins of the Annexin family. The graph shows the ELISA signal obtained in “relative fluorescence units” (RFV) on the VIDAS® for each of the TGC44/5C5B10 and TGC44/13A12G4H2 test formats and for each Annexin indicated on the x-axis. For comparison, under the experimental conditions employed, Annexin A3 served to obtain a signal higher than 6000 RFV with each of the test formats.

FIG. 8 shows the sensorgrams obtained for each of the 6 monoclonal antibodies characterised with Biacore T100. The graphs show the resonance signal measured in “resonance units” (RU) as a function of time. Each curve of a graph represents all the measurements taken for a given Annexin A3 concentration. For each antibody, 9 dilutions between 0 and 64 nM of Annexin A3 were analysed and are shown.

FIG. 9 shows the Western blot analysis of the exosomes purified (ExoQuick treatment) from serums of 2 patients suffering from prostate cancer. The membranes were incubated with either an anti-CD63 antibody or with an anti-ANXA3 antibody.

### EXAMPLES

### Example 1

### Detection of Human Annexin A3 by ELISA in the Serum of Patients Having a Prostate Cancer

Obtaining the Monoclonal Antibodies

The immunisation experiments were conducted on female BALB/c (H-2^{d}) mice between six and eight weeks of age at the time of the first immunisation. The native human Annexin A3 protein was purchased from Arodia Arotech Diagnostic™ (Cat No. 25592), and was purified using human neutrophils. This protein was mixed volume per volume with Freund's adjuvant (Sigma Aldrich™), prepared in the form of a water-in-oil emulsion and known to display good immunogenic potential. The mice received three successive doses of 10 μg of immunogen at zero, two and four weeks. All the injections were subcutaneous. The first injection was performed in a mixture with Freund's complete adjuvant, and the following two in a mixture with Freund's incomplete adjuvant. Between J50 and J70 after the first injection, the humoral responses were restimulated with an intravenous injection of 100 μg of native protein.

In order to monitor the appearance of the antibodies, blood samples were taken regularly from the mice. The presence of anti-ANXA3 antibodies was tested using an ELISA. The protein under investigation was used for capture (1 μg/well), after saturation the antigen was reacted with various dilutions of serums to be tested (incubation at 37° C. for 1 h). The specific antibodies present in the serum were visualised by AffiniPure™ mouse anti-IgG goat antibody combined with alkaline phosphotase (H+L, Jackson Immunoresearch, Cat no. 115-055-146), which bound to the desired antibodies (0.1 μg/well).

Three days after the last injection, one of the immunised mice was sacrificed; the blood and spleen were sampled. The splenocytes obtained from the spleen were cultured with Sp2/0-Ag14 myeloma cells to fuse and to become immortal, following the procedure described by After an incubation period of 12-14 days, the hybridoma supernatants obtained were screened to determine the presence of anti-ANXA3 antibodies using the ELISA test described in the previous paragraph. The immunogen (native ANXA3), the recombinant Annexin A3 produced in E. coli, and various human cells expressing ANXA3, were used in succession to screen the hybridoma supernatants. The positive hybridoma colonies were subcloned twice using the limited dilution technique, well known to a person skilled in the art.

The following anti-Annexin A3 monoclonal antibodies were thus obtained: 5C5B10, 13A12G4H2, 9C6B4, 6D9D10, 1F10A6.

Selection of Anti-ANXA3 Monoclonal Antibodies for Assay by Immuno Test of ANXA3

The complementarity of the various anti-ANXA3 antibodies obtained as described above and the TGC42, TGC43 and TGC44 antibodies described in Patent Application WO 2010/034825 was analysed using as the antigen the native human ANXA3 (immunogen) diluted in PBS buffer, forming a sandwich-type immuno test. This type of assay can be carried out with a microplate, in an automated or manual manner, or even by using immunoanalysis robots like the VIDAS® (bioMérieux).

The reagents of the VIDAS® HBs Ag Ultra kit (bioMérieux, Cat no. 30315) were used, such as those described in the corresponding manual (ref. 11728 D-FR-2005/5), and modified as follows:
Clones were sensitised with one of the capture antibodies to be tested, TGC42, TGC43, TGC44 or 9C6B4 in a concentration of 10 μg/mL.
The content of the second well of the HBs Ag Ultra cartridge was replaced by 300 μL of visualisation antibody to be tested (5C5B10, 13A12G4H2, 9C6B4, 6D9D10, 1F10A6, TGC42, TGC43, TGC44), coupled with biotin, diluted to 1 μg/mL in the buffer of the second well of the VIDAS® HBs Ag Ultra kit (well X1) containing goat serum and 1 g/l sodium azodide.
The native human ANXA3 protein was tested diluted to 100, 25 and 3 ng/mL in PBS buffer. The sample was deposited (150 μL) in the first well (well X0) of the HBs Ag Ultra cartridge.
The ELISA reaction was performed using the VIDAS® robot and the protocol described for the HBs Ag Ultra test.
The results were obtained in the form of raw values after subtraction of background noise. The signal is in RFV (“relative fluorescence value”).

Table 2 below shows the results obtained (RFV signal), with the various antibody combinations used in capture or detection, on three dilutions of native human Annexin A3 purified of the neutrophils (100, 25 and 3 ng/mL). The dilution 0 ng/mL corresponds to the PBS buffer control, without Annexin A3.

TABLE 2

| Biotinylated visualisation | | | | | |
|---|---|---|---|---|---|
| antibodies | | | | | |
| | ANXA3 | Capture antibodies | | | |
| Clone | dilution ng/mL | TGC42 | TGC43 | TGC44 | 9C6B4 |
| TGC42 | 0 | — | 22 | 11 | 17 |
| | 3 | — | 44 | 20 | 78 |
| | 25 | — | 238 | 89 | 500 |
| | 100 | — | 859 | 297 | 1837 |
| TGC43 | 0 | 106 | — | 39 | 63 |
| | 3 | 111 | — | 36 | 82 |
| | 25 | 144 | — | 56 | 257 |
| | 100 | 232 | — | 86 | 914 |
| TGC44 | 0 | 21 | 9 | — | 12 |
| | 3 | 32 | 19 | — | 19 |
| | 25 | 98 | 89 | — | 74 |
| | 100 | 303 | 309 | — | 261 |
| 9C6B4 | 0 | 51 | 42 | 33 | — |
| | 3 | 65 | 122 | 39 | — |
| | 25 | 201 | 744 | 117 | — |
| | 100 | 590 | 3525 | 357 | — |
| 5C5B10 | 0 | 20 | 12 | 93 | 9 |
| | 3 | 5244 | 3669 | 5870 | 39 |
| | 25 | 10480 | 9874 | 10429 | 249 |
| | 100 | 11310 | 11095 | 11168 | 1139 |
| 13A12G4H2 | 0 | 26 | 21 | 18 | 19 |
| | 3 | 2298 | 676 | 3733 | 23 |
| | 25 | 9655 | 8468 | 10208 | 21 |
| | 100 | 11380 | 10995 | 11332 | 21 |
| 6D9D10 | 0 | 12 | 23 | 16 | 9 |
| | 3 | 11 | 23 | 14 | 9 |
| | 25 | 12 | 23 | 18 | 19 |
| | 100 | 18 | 41 | 43 | 27 |
| 1F10A6 | 0 | 45 | 9 | 14 | 14 |
| | 3 | 159 | 207 | 194 | 17 |
| | 25 | 4014 | 6728 | 5680 | 13 |
| | 100 | 10489 | 10931 | 10688 | 15 |

Table 3 below shows the results obtained described in Table 2, but with a different reading grid, to facilitate analysis and interpretation:

If RFV at 3 ng/mL <1000 then “−”

If RFV at 3 ng/mL >1000 then “+”

If RFV at 25 ng/mL >3000 then “++”

If (RFV at 100 ng/mL >9000) and (3000<RFV at 25 ng/mL <9000) then “+++”

If RFV at 100 ng/mL and 25 ng/mL >9000 then “++++”

TABLE 3

| Capture | Biotinylated detection *mAb | | | | | | | |
|---|---|---|---|---|---|---|---|---|
| *mAb | TGC42 | TGC43 | TGC44 | 9C6B4 | 5CSB10 | 13A12G4H2 | 1F10A6 | 6D9D10 |
| TGC42 | − | − | − | − | ++++ | ++++ | +++ | − |
| TGC43 | − | − | − | + | ++++ | +++ | +++ | − |
| TGC44 | − | − | − | − | ++++ | ++++ | +++ | − |
| 9C6B4 | + | − | − | − | + | − | − | − |

mAb = monoclonal antibodies

As it appears from the table above, there are 9 combinations of complementary monoclonal antibodies which serve to perform a sandwich-type ELISA assay of ANXA3, with a very satisfactory signal dynamic range (“+++” and “++++” pairs). Other combinations of complementary monoclonal antibodies are feasible, that is to say 9C6B4/TGC42, 9C6B4/TGC43 and 9C6B4/5C5B10, but these solutions are not sufficiently robust from the analytical standpoint and do not have sufficient analytical sensitivity to be used in complex biological fluids like serum or urine, for example.

For these selection experiments carried out by diluting purified human Annexin A3 in PBS buffer the monoclonal antibodies TGC42, TGC43 and TGC44 have, in capture, equivalent performance with regard to human Annexin A3.

In detection, the monoclonal antibodies 5C5B10 and 13A12G4H2 likewise possess equivalent performance with regard to human Annexin A3, whereas the monoclonal antibody 1F10A6 yields satisfactory but weaker signals than with the monoclonal antibodies 5C5B10 and 13A12G4H2.

The experiments presented in Example 7 serve to demonstrate that all three monoclonal antibodies TGC42, TGC43 and TGC44 recognise the same region of Annexin A3. In the same way, the monoclonal antibodies 13A12G4H2 and 1F10A6 also recognise the same epitope but one that is distinct from the first. As to the antibody 5C5B10, it recognises a third epitope distinct from the first two. Thus, the various types of ELISA assay which serve to detect ANXA3 are divided into two groups. Group 1 corresponds to combinations of the capture antibodies TGC42, TGC43 or TGC44 with the clones 13A12G4H2 or 1F10A6 used in detection. Group 2 corresponds to combinations of capture antibodies TGC42, TGC43 or TGC44 with the monoclonal 5C5B10 used in detection.

Detection of Human Annexin A3 in the Serum of Subjects Having a Prostate Cancer

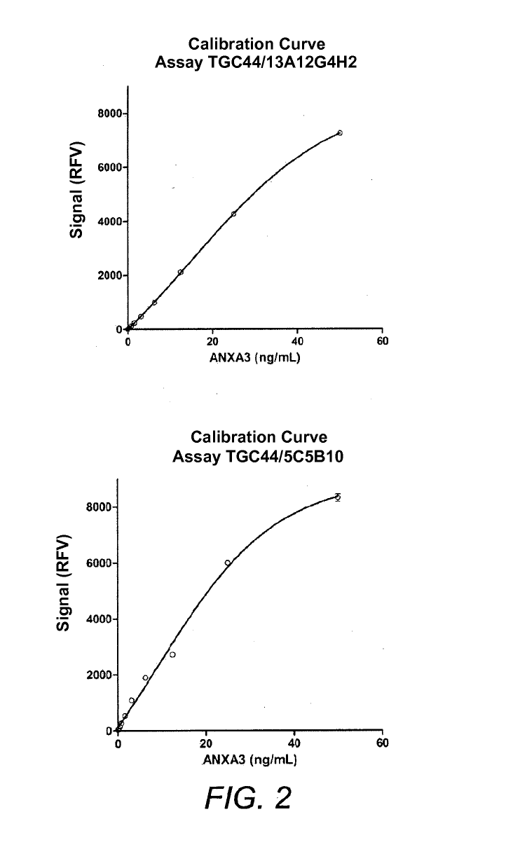

Whole blood was sampled in 56 patients using the S-Monovette sampling system (Sarstedt, Cat. No. 02.1388) and treated following the manufacturer's instructions to obtain serum. These 56 subjects all had a proven prostate cancer (CaPro), whose diagnosis was confirmed by histological analysis on biopsy subsequently. The ANXA3 contained in these 56 serum samples was quantified with an ELISA group 1 determination (capture TGC44, detection 13A12G4H2) and an ELISA group 2 determination (capture TGC44, detection 5C5B10). For information, the biotinylated detection antibodies were diluted to 0.3 μg/mL for the monoclonal 5C5B10 and to 2.5 ng/mL for the monoclonal 13A12G4H2. For each ELISA assay, a calibration curve was plotted by assaying a concentration range of purified native human Annexin A3 protein (Arodia™) diluted in an appropriate buffer. The serums of animal or human origin potentially contained in this buffer were depleted beforehand of any endogenous Annexin A3 which they contained, in order to avoid giving a false result of the assay. Alternatively, batches of animal-origin serum containing only very low quantities of endogenous Annexin A3 were used. The calibration curve was plotted with the concentration on the x-axis and the measured signal by the VIDAS® in RFV on the y-axis (FIG. 2). The concentration of ANXA3 present in the serum sample was calculated, whenever possible, by interpolating the concentration corresponding to the RFV signal read by the VIDAS®, using mathematical models of nonlinear regression as a third-order polynomial or a 4-PL model, well known to a person skilled in the art. The results are given in Table 4 below.

TABLE 4

| | |
|---|---|
| | Assay |
| Assay | VTGC44/ |
| TGC44/5C5B10 | 13A12G4H2 |

| Sample | Signal | Dose | Signal | Dose |
|---|---|---|---|---|
| Code | (RFV) | (ng/mL) | (RFV) | (ng/mL) |
| CaPro01 | 153 | <0.5 | 914 | 5.8 |
| CaPro02 | 38 | <0.5 | 380 | 2.7 |
| CaPro03 | 47 | <0.5 | 779 | 5.0 |
| CaPro04 | 38 | <0.5 | 572 | 3.8 |
| CaPro05 | 64 | <0.5 | 2431 | 14.1 |
| CaPro06 | 41 | <0.5 | 452 | 3.1 |
| CaPro07 | 71 | <0.5 | 2095 | 12.2 |
| CaPro08 | 44 | <0.5 | 619 | 4.1 |
| CaPro09 | 66 | <0.5 | 1493 | 9.0 |
| CaPro10 | 39 | <0.5 | 401 | 2.8 |
| CaPro11 | 42 | <0.5 | 736 | 4.8 |
| CaPro12 | 60 | <0.5 | 2000 | 11.7 |
| CaPro13 | 65 | <0.5 | 2419 | 14.0 |
| CaPro14 | 51 | <0.5 | 1121 | 6.9 |
| CaPro15 | 46 | <0.5 | 566 | 3.8 |
| CaPro16 | 58 | <0.5 | 852 | 5.5 |
| CaPro17 | 44 | <0.5 | 401 | 2.8 |
| CaPro18 | 62 | <0.5 | 1039 | 6.5 |
| CaPro19 | 56 | <0.5 | 983 | 6.2 |
| CaPro20 | 70 | <0.5 | 601 | 4.0 |
| CaPro21 | 55 | <0.5 | 954 | 6.0 |
| CaPro22 | 43 | <0.5 | 595 | 4.0 |
| CaPro23 | 43 | <0.5 | 680 | 4.5 |
| CaPro24 | 55 | <0.5 | 1324 | 8.1 |
| CaPro25 | 40 | <0.5 | 564 | 3.8 |
| CaPro26 | 45 | <0.5 | 758 | 4.9 |
| CaPro27 | 78 | <0.5 | 3106 | 17.9 |
| CaPro28 | 60 | <0.5 | 205 | 1.5 |
| CaPro29 | 57 | <0.5 | 1583 | 9.5 |
| CaPro30 | 63 | <0.5 | 1583 | 9.5 |
| CaPro31 | 85 | <0.5 | 583 | 3.9 |
| CaPro32 | 47 | <0.5 | 703 | 4.6 |
| CaPro33 | 50 | <0.5 | 331 | 2.3 |
| CaPro34 | 43 | <0.5 | 361 | 2.5 |
| CaPro35 | 46 | <0.5 | 1316 | 8.0 |
| CaPro36 | 42 | <0.5 | 513 | 3.5 |
| CaPro37 | 52 | <0.5 | 872 | 5.6 |
| CaPro38 | 36 | <0.5 | 100 | 0.6 |
| CaPro39 | 44 | <0.5 | 598 | 4.0 |
| CaPro40 | 33 | <0.5 | 643 | 4.3 |
| CaPro41 | 93 | <0.5 | 2891 | 16.7 |
| CaPro42 | 54 | <0.5 | 141 | 1.0 |
| CaPro43 | 54 | <0.5 | 829 | 5.3 |
| CaPro44 | 41 | <0.5 | 761 | 4.9 |
| CaPro45 | 56 | <0.5 | 742 | 4.8 |
| CaPro46 | 53 | <0.5 | 1148 | 7.1 |
| CaPro47 | 44 | <0.5 | 382 | 2.7 |
| CaPro48 | 48 | <0.5 | 490 | 3.3 |
| CaPro49 | 72 | <0.5 | 1422 | 8.6 |
| CaPro50 | 47 | <0.5 | 535 | 3.6 |
| CaPro51 | 88 | <0.5 | 1414 | 8.5 |
| CaPro52 | 72 | <0.5 | 2866 | 16.5 |
| CaPro53 | 43 | <0.5 | 315 | 2.2 |
| CaPro54 | 83 | <0.5 | 1777 | 10.5 |
| CaPro55 | 53 | <0.5 | 1315 | 8.0 |
| CaPro56 | 48 | <0.5 | 588 | 3.9 |

It is possible to measure serum ANXA3 concentrations of the order of ng/mL and of tens of ng/mL using the ELISA TGC44/13A12G4H2 assay (group 1). Unexpectedly, the signals obtained in the same serum samples with the TGC44/5C5B10 assay (group 2) are very weak and virtually indistinguishable from the background noise. Moreover, a calibration curve using a 4-PL model did not allow conversion of these signals to doses, which is why the dose is reported as “0.5 ng/mL” in Table 4, which is the assay quantification limit under the conditions of use indicated here. The calibration curve achieved with purified native human ANXA3 indicates that the TGC44/5C5B10 assay has the capacity to detect the molecule when its concentration is of the order of ng/mL. However, despite a satisfactory analytical sensitivity (quantification limit about 0.5 ng/mL) and satisfactory analytical specificity (see Example 3), the TGC44/5C5B10 assay does not make it possible to assay the ANXA3 present in the complex biological medium consisting of the serum.

These results are extremely surprising and demonstrate a hitherto unsuspected complexity of the serum ANXA3. The difference between the two ELISA tests stems from the detection antibody. In fact, the monoclonal antibody 5C5B10 serves to detect the purified ANXA3 captured by TGC44, but not the ANXA3 captured directly from the serum by TGC44. The 13A12GH2 antibody (and also the 1F10A6 antibody) serves to detect the captured ANXA3, whether it is the purified protein or the protein present in the serum.

### Example 2

### Detection of Human Annexin A3 by ELISA in the Serum of Patients Having a Colorectal Cancer

The whole blood was sampled in 39 subjects using Vacutainer® BD tubes (BD, Cat. No. 367614) and treated according to the manufacturer's instructions to obtain serum. These 39 subjects all had a proven colorectal cancer (CCR), whose diagnosis was confirmed by histological examination on surgical specimens. The ANXA3 contained in these 39 serum samples was quantified with an ELISA group 1 assay (capture TGC44, detection 13A12G4H2) and an ELISA group 2 assay (capture TGC44, detection 5C5B10). For information, the biotinylated detection antibodies were diluted to 0.15 μg/mL for the monoclonal 5C5B10 and to 2 ng/mL for the monoclonal 13A12G4H2. For each ELISA assay, a calibration curve was plotted by assaying a concentration range of purified native human Annexin A3 protein (Arodia). The calibration curve was plotted with the concentration on the x-axis and the measured signal by the VIDAS® in RFV on the y-axis. The concentration of ANXA3 present in the serum sample was calculated, whenever possible, by interpolating the concentration corresponding to the RFV signal read by the VIDAS®, using mathematical models of nonlinear regression as a third-order polynomial or a 4-PL model, well known to a person skilled in the art. The results are given in Table 5 below.

TABLE 5

| | |
|---|---|
| | Assay |
| Assay | TGC44/ |
| TGC44/5C5B10 | 13A12G4H2 |

| Sample | Signal | Dose | Signal | Dose |
|---|---|---|---|---|
| Code | (RFV) | (ng/mL) | (RFV) | (ng/mL) |
| CCR01 | 59 | <0.5 | 5358 | 20.5 |
| CCR02 | 20 | <0.5 | 889 | 3.9 |
| CCR03 | 79 | <0.5 | 3648 | 13.1 |
| CCR04 | 289 | 0.9 | 2120 | 7.9 |
| CCR05 | 68 | <0.5 | 1526 | 6.0 |
| CCR06 | 49 | <0.5 | 1127 | 4.7 |
| CCR07 | 196 | 0.5 | 809 | 3.6 |
| CCR08 | 24 | <0.5 | 346 | 1.8 |
| CCR09 | 86 | <0.5 | 3692 | 13.3 |
| CCR10 | 58 | <0.5 | 2806 | 10.2 |
| CCR11 | 77 | <0.5 | 750 | 3.4 |
| CCR12 | 46 | <0.5 | 1533 | 6.0 |
| CCR13 | 102 | <0.5 | 210 | 1.1 |
| CCR14 | 42 | <0.5 | 2338 | 8.6 |
| CCR15 | 149 | <0.5 | 5744 | 22.5 |
| CCR16 | 32 | <0.5 | 141 | 0.7 |
| CCR17 | 98 | <0.5 | 1327 | 5.4 |
| CCR18 | 114 | <0.5 | 4228 | 15.4 |
| CCR19 | 69 | <0.5 | 4568 | 16.8 |
| CCR20 | 161 | <0.5 | 3071 | 11.1 |
| CCR21 | 65 | <0.5 | 1469 | 5.8 |
| CCR22 | 40 | <0.5 | 764 | 3.5 |
| CCR23 | 106 | <0.5 | 1983 | 7.5 |
| CCR24 | 53 | <0.5 | 5427 | 20.8 |
| CCR25 | 48 | <0.5 | 2743 | 10.0 |
| CCR26 | 136 | <0.5 | 2445 | 9.0 |
| CCR27 | 238 | 0.7 | 1382 | 5.5 |
| CCR28 | 38 | <0.5 | 1221 | 5.0 |
| CCR29 | 33 | <0.5 | 1604 | 6.3 |
| CCR30 | 85 | <0.5 | 3063 | 11.0 |
| CCR31 | 46 | <0.5 | 2176 | 8.1 |
| CCR32 | 42 | <0.5 | 1004 | 4.3 |
| CCR33 | 92 | <0.5 | 2383 | 8.8 |
| CCR34 | 35 | <0.5 | 1567 | 6.1 |
| CCR35 | 88 | <0.5 | 620 | 2.9 |
| CCR36 | 166 | <0.5 | 6897 | 30.3 |

In the serum of patients having a colorectal cancer, it is possible to detect and quantify the Annexin A3 using the ELISA TGC44/13A12G4H2 assay (group 1). The ELISA TGC44/5C5B10 assay (group 2) makes the signals very weak, lower than or at the quantifiable limit. It is therefore not possible to use the TGC44/5C5B10 format to quantify the Annexin A3 present in the serum of patients having a colorectal cancer.

### Example 3

### Detection of Human Annexin A3 by ELISA in the Serum of Patients Having a Lung Cancer

The Annexin A3 assays were performed on serum samples taken from 33 patients having a bronchopulmonary cancer (CaPou) confirmed by the procedure described in Example 2. The results are given in Table 6 below.

TABLE 6

| | |
|---|---|
| Assay | Assay |
| TGC44/5C5B10 | TGC44/13A12G4H2 |

| Sample | Signal | Dose | Signal | Dose |
|---|---|---|---|---|
| Code | (RFV) | (ng/mL) | (RFV) | (ng/mL) |
| CaPou01 | 235 | 0.7 | 2347 | 8.7 |
| CaPou02 | 619 | 2.0 | 928 | 4.0 |
| CaPou03 | 1219 | 4.1 | 1453 | 5.8 |
| CaPou04 | 58 | <0.5 | 729 | 3.3 |
| CaPou05 | 253 | 0.7 | 5505 | 21.2 |
| CaPou06 | 127 | <0.5 | 514 | 2.5 |
| CaPou07 | 127 | <0.5 | 4433 | 16.2 |
| CaPou08 | 320 | 1.0 | 2953 | 10.7 |
| CaPou09 | 223 | 0.6 | 6373 | 26.4 |
| CaPou10 | 329 | 1.0 | 9056 | 61.0 |
| CaPou11 | 864 | 2.9 | 7703 | 37.9 |
| CaPou12 | 125 | <0.5 | 3115 | 11.2 |
| CaPou13 | 354 | 1.1 | 8069 | 42.4 |
| CaPou14 | 385 | 1.2 | 8268 | 45.3 |
| CaPou15 | 428 | 1.4 | 8060 | 42.3 |
| CaPou16 | 333 | 1.0 | 4396 | 16.1 |
| CaPou17 | 185 | <0.5 | 1770 | 6.8 |
| CaPou18 | 670 | 2.2 | 1278 | 5.2 |
| CaPou19 | 206 | 0.6 | 5219 | 19.8 |
| CaPou20 | 40 | <0.5 | 615 | 2.9 |
| CaPou21 | 330 | 1.0 | 6404 | 26.6 |
| CaPou22 | 110 | <0.5 | 860 | 3.8 |
| CaPou23 | 561 | 1.8 | 932 | 4.0 |
| CaPou24 | 50 | <0.5 | 573 | 2.7 |
| CaPou25 | 166 | <0.5 | 1087 | 4.6 |
| CaPou26 | 49 | <0.5 | 844 | 3.7 |
| CaPou27 | 82 | <0.5 | 534 | 2.6 |
| CaPou28 | 97 | <0.5 | 1288 | 5.2 |
| CaPou29 | 121 | <0.5 | 652 | 3.0 |
| CaPou30 | 131 | <0.5 | 2969 | 10.7 |
| CaPou31 | 99 | <0.5 | 503 | 2.5 |
| CaPou32 | 207 | 0.6 | 1668 | 6.5 |
| CaPou33 | 210 | 0.6 | 3770 | 13.6 |

In the serum of patients having a bronchopulmonary cancer, it is possible to detect and quantify the Annexin A3 using the ELISA TGC44/13A12G4H2 assay (group 1). The doses obtained on this series of patients range from 2.5 to 61 ng/mL. The ELISA TGC44/5C5B10 assay (group 2) makes the signals much weaker, with 15 samples out of 33 for which the doses are unquantifiable because too low. In this series of patients, the TGC44/5C5B10 format makes the doses, at about 1-2 ng/mL, much too low in comparison with the doses obtained with the TGC44/13A12G4H2 format. It is therefore not possible to use the TGC44/5C5B10 format to quantify the Annexin A3 present in the serum of patients having a lung cancer.

### Example 4

### Detection of Human Annexin A3 by ELISA in the Serum of Patients Having a Chronic Inflammatory Pathology

The Annexin A3 assays were performed on serum samples taken from 10 patients having a rheumatoid polyarthritis (PR, chronic inflammatory degenerative disease of the joints) and from 21 patients having Crohn's disease (MC, chronic inflammatory intestinal disease) according to the procedures described in Example 2. The results are given in Table 7 below.

TABLE 7

| | |
|---|---|
| | Assay |
| Assay TGC44/5C5B10 | TGC44/13A12G4H2 |

| Sample | Signal | Dose | Signal | Dose |
|---|---|---|---|---|
| Code | (RFV) | (ng/mL) | (RFV) | (ng/mL) |
| PR01 | 182 | <0.5 | 3060 | 11.0 |
| PR02 | 596 | 2.0 | 6323 | 26.1 |
| PR03 | 577 | 1.9 | 4358 | 15.9 |
| PR04 | 497 | 1.6 | 2482 | 9.1 |
| PR05 | 540 | 1.8 | 4862 | 18.1 |
| PR06 | 336 | 1.1 | 3226 | 11.6 |
| PR07 | 749 | 2.5 | 6067 | 24.4 |
| PR08 | 248 | 0.7 | 5669 | 22.1 |
| PR09 | 688 | 2.3 | 8630 | 51.4 |
| PR10 | 285 | 0.9 | 6369 | 26.4 |
| MC01 | 77 | <0.5 | 2279 | 8.4 |
| MC02 | 101 | <0.5 | 4034 | 14.6 |
| MC03 | 47 | <0.5 | 7029 | 31.4 |
| MC04 | 165 | <0.5 | 4575 | 16.8 |
| MC05 | 85 | <0.5 | 1947 | 7.4 |
| MC06 | 64 | <0.5 | 3924 | 14.2 |
| MC07 | 96 | <0.5 | 4267 | 15.5 |
| MC08 | 97 | <0.5 | 1797 | 6.9 |
| MC09 | 197 | 0.5 | 5052 | 19.0 |
| MC10 | 32 | <0.5 | 1004 | 4.3 |
| MC11 | 246 | 0.7 | 3095 | 11.2 |
| MC12 | 99 | <0.5 | 2972 | 10.7 |
| MC13 | 52 | <0.5 | 3938 | 14.2 |
| MC14 | 158 | <0.5 | 7637 | 37.2 |
| MC15 | 297 | 0.9 | 1640 | 6.4 |
| MC16 | 104 | <0.5 | 7952 | 40.9 |
| MC17 | 254 | 0.7 | 2510 | 9.2 |
| MC18 | 58 | <0.5 | 2570 | 9.4 |
| MC19 | 46 | <0.5 | 3027 | 10.9 |
| MC20 | 41 | <0.5 | 1947 | 7.4 |
| MC21 | 129 | <0.5 | 3320 | 11.9 |

In the serum of patients having rheumatoid polyarthritis or Crohn's disease, it is possible to detect and quantify the Annexin A3 using the ELISA TGC44/13A12G4H2 assay (group 1). The doses obtained on this series of patients range from 4.3 to 51.4 ng/mL. The ELISA TGC44/5C5B10 assay (group 2) makes the signals much weaker, with 18 samples out of 31 for which the dose is unquantifiable because too low. In this series of patients, the doses produced by the TGC44/5C5B10 format do not exceed 2.5 ng/mL, much too low in comparison with the doses obtained with the TGC44/13A12G4H2 format. It is therefore not possible to use the TGC44/5C5B10 format to quantify the Annexin A3 present in the serum of patients having a chronic inflammatory disease.

### Example 5

### Detection of Human Annexin A3 by ELISA in the Serum of Patients Having a Sepsis Risk

The Annexin A3 assays were performed on samples of serum taken from 24 patients having sepsis risk according to the procedures described in Example 2. Procalcitonin (PCT) is a specific and early marker of bacterial infection. During localised infections, the PCT rate can reach 0.1 ng/mL and it exceeds 1-2 ng/mL with generalised infections. The serums included in the series presented in Table 8 have PCT rates of between 0.3 and 10.3 ng/mL (rate determined with the BRAHMS™ KRYPTOR™ PCT kit).

TABLE 8

| | |
|---|---|
| | Assay |
| Assay TGC44/5C5B10 | TGC44/13A12G4H2 |

| Sample | Signal | Dose | Signal | Dose |
|---|---|---|---|---|
| Code | (REV) | (ng/mL) | (REV) | (ng/mL) |
| PCT01 | 176 | <0.5 | 370 | 1.9 |
| PCT02 | 352 | 1.1 | 3786 | 13.6 |
| PCT03 | 225 | 0.6 | 902 | 3.9 |
| PCT04 | 2630 | 9.1 | 7797 | 39.0 |
| PCT05 | 719 | 2.4 | 5204 | 19.7 |
| PCT06 | 1244 | 4.2 | 5574 | 21.6 |
| PCT07 | 534 | 1.8 | 5773 | 22.7 |
| PCT08 | 137 | <0.5 | 1681 | 6.5 |
| PCT09 | 1365 | 4.6 | 9101 | 62.2 |
| PCT10 | 815 | 2.7 | 8800 | 54.9 |
| PCT11 | 563 | 1.9 | 6926 | 30.5 |
| PCT12 | 331 | 1.0 | 6579 | 27.8 |
| PCT13 | 405 | 1.3 | 2054 | 7.7 |
| PCT14 | 458 | 1.5 | 3276 | 11.8 |
| PCT15 | 2003 | 6.8 | 5609 | 21.8 |
| PCT16 | 303 | 0.9 | 3976 | 14.4 |
| PCT17 | 218 | 0.6 | 3298 | 11.9 |
| PCT18 | 738 | 2.5 | 7627 | 37.1 |
| PCT19 | 465 | 1.5 | 7374 | 34.5 |
| PCT20 | 527 | 1.7 | 798 | 3.6 |
| PCT21 | 236 | 0.7 | 1568 | 6.1 |
| PCT22 | 200 | 0.5 | 1876 | 7.1 |
| PCT23 | 601 | 2.0 | 5755 | 22.6 |
| PCT24 | 3405 | 12.3 | 9227 | 65.8 |

In the serum of patients having a bacterial infection, it is possible to detect and quantify the Annexin A3 using the ELISA TGC44/13A12G4H2 assay (group 1). The doses obtained on this series of patients range from 1.9 to 65.8 ng/mL. The ELISA TGC44/5C5B10 assay (group 2) makes the signals much weaker and the doses remain low in comparison with the doses obtained with the TGC44/13A12G4H2 format. It does not appear useful to use the TGC44/5C5B10 format to quantify the Annexin A3 present in the serum of patients having a sepsis risk.

### Example 6

### Detection of Human Annexin A3 by ELISA in the Serum of Patients Having Diabetes

The Annexin A3 assays were performed on samples of serum taken from 6 patients having diabetes, according to the procedures described in Example 2. The results are given in Table 9 below.

TABLE 9

| | |
|---|---|
| | Assay |
| Assay TGC44/5C5B10 | TGC44/13A12G4H2 |

| Sample | Signal | Dose | Signal | Dose |
|---|---|---|---|---|
| Code | (RFV) | (ng/mL) | (RFV) | (ng/mL) |
| Dia01 | 51 | <0.5 | 444 | 2.2 |
| Dia02 | 231 | 0.7 | 1664 | 6.5 |
| Dia03 | 44 | <0.5 | 585 | 2.8 |
| Dia04 | 171 | <0.5 | 3263 | 11.7 |
| Dia05 | 213 | 0.6 | 3495 | 12.6 |
| Dia06 | 289 | 0.9 | 2564 | 9.4 |

In the serum of patients having diabetes, it is possible to detect and quantify the Annexin A3 using the ELISA TGC44/13A12G4H2 assay (group 1). The ELISA TGC44/5C5B10 assay (group 2) makes the signals very weak, lower than or at the quantifiable limit. It is therefore not possible to use the TGC44/5C5B10 format to quantify the Annexin A3 present in the serum of patients having diabetes.

### Example 7

### Determination of Epitopes Recognised by the Anti-ANXA3 Monoclonal Antibodies

Expression of 4 Annexin Repeat Domains of Human Annexin A3 in Recombinant Form and Determination of the Repeat Domains Recognised by the Monoclonal Antibodies.

Like all the members of the Annexin family, Annexin A3 contains in its protein sequence repeat domains called “Annexin repeat domains”. These repeat domains are 4 in number and characterise the family. In order to determine the repeat domain recognised by each of the monoclonal antibodies, these domains were expressed in recombinant form. A sequence of 8 histidines was added to the N-terminus part of each domain to allow purification by metal-chelate affinity chromatography. Table 10 gives the protein sequences of the recombinant structures serving to express each of the repeat domains in an isolated manner.

TABLE 10

| Do- | | Ex- | |
|---|---|---|---|
| main | Real ^{a} | pressed ^{b} | Protein sequence |
| D1 | 27-87 | 19-89 | MGHHHHHHHHSPSVDAEAIQKAIR |
| | | | GIGTDEKMLISILTERSNAQRQLI |
| | | | VKEYQAAYGKELKDDLKGDLSGHF |
| | | | EHLMVALVT |
| | | | (SEQ ID NO: 6) |
| D2 | 99-159 | 92-160 | MGHHHHHHHHAVFDAKQLKKSMKG |
| | | | AGTNEDALIEILTTRTSRQMKDIS |
| | | | QAYYTVYKKSLGDDISSETSGDFR |
| | | | KALLTLA |
| | | | (SEQ ID NO: 7) |
| D3 | 183-243 | 171-245 | MGHHHHHHHHDEHLAKQDAQILYK |
| | | | AGENRWGTDEDKFTEILCLRSFPQ |
| | | | LKLTFDEYRNISQKDIVDSIKGEL |
| | | | SGHFEDLLLAIVN |
| | | | (SEQ ID NO: 8) |
| D4 | 258-318 | 252-323 | MGHHHHHHHHAFLAERLHRALKGI |
| | | | GTDEFTLNRIMVSRSEIDLLDIRT |
| | | | EFKKHYGYSLYSAIKSDTSGDYEI |
| | | | TLLKICGGDD |
| | | | (SEQ ID NO: 9) |

^{a} Real: Extent of the repeat domain, from the
first to the last amino acid, according to the
UniProtKB database (http://www.uniprot.org).
^{b} Expressed: Extent of the structure containing
the repeat domain, numbering of amino acids
according to UniProtKB. The structures contain
a few additional amino acids on the N and C
terminus sides of the domain, to avoid inter-
rupting the alpha helices and to enable them
to form.

The nucleic acid sequences corresponding to the protein sequences of the domains D1, D2, D3 and D4 were obtained by chemical synthesis performed by Geneart. These nucleic sequences were optimised to promote the expression of the proteins in Escherichia coli. The DNA fragments were cloned between the Nco I and Xba I sites of the pMRCH79 procaryote expression vector (derivative of pMR78, bioMérieux). The plasmids thus obtained were converted to BL21 (DE3) bacteria (Stratagene). The cultures for producing the various domains are prepared at 37° C. with stirring in 2-YT medium (Invitrogen). Induction is carried out with 0.5 mM IPTG (isopropyl beta-1-thiogalactosidase). The bacterial pellets are directly taken up in the buffer of Novex NuPAGE gel samples (Invitrogen) following the procedure provided with the gels, in reduced conditions. The proteins are separated in NuPAGE Novex Bis-Tris 4-12% gel. The Western blot analysis of the reactivity of the monoclonal antibodies for the various domains of Annexin A3 was performed using a chemiluminescent substrate, by the method described in Patent Application WO 2009/019365 for example, well known to a person skilled in the art. The test antibodies were used in a dilution of 10 μg/mL. The exposure time was 100 seconds, unless otherwise indicated.

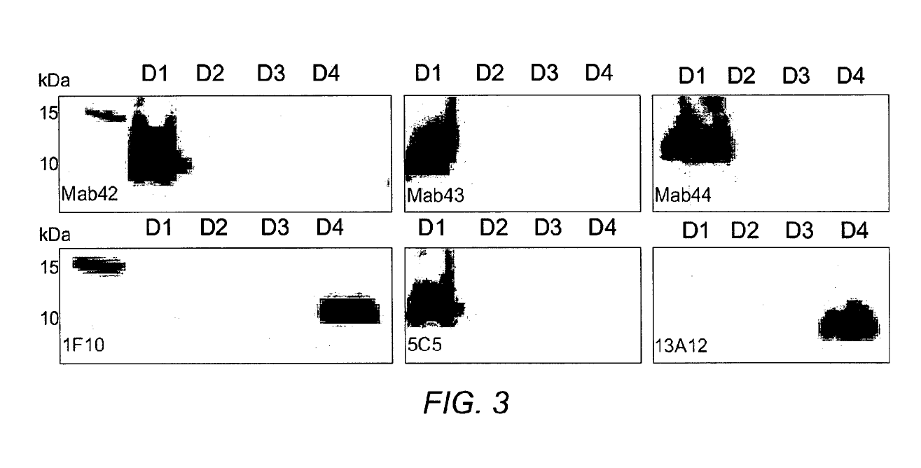

Each anti-ANXA3 antibody was tested with the recombinants expressing the 4 repeat domains of human ANXA3; the results of this Western blot analysis are given in FIG. 3. The monoclonal antibodies TGC42, TGC43, TGC44 and 5C5B10 are specific to domain D1. The monoclonal antibodies 13A12G4H2 and 1F10A6 are directed against domain D4. As to the antibodies 9C6D4 and 6D9D10, they do not recognise any of the 4 repeat domains, very probably indicating that their epitopes are located outside the repeat domains of ANXA3.

Fine Analysis of the Epitopes Recognised by the Anti-ANXA3 Antibodies

The epitopes were determined by the Spotscan technique after Frank and Daring which is described in detail in Patent Application WO 2009/019365. For this purpose, the entire protein sequence of Annexin A3 was reproduced on a nitrocellulose membrane in the form of peptides of 12 overlapping amino acids, offset by 2 amino acids. Then, in a second synthesis, the ANXA3 sequence was reproduced in the form of peptides of 15 overlapping amino acids, offset by one amino acid. The immunoreactivity of these overlapping peptide membranes was tested with the anti-ANXA3 antibodies.

Thus, it was possible to obtain a finer delimitation of the epitopes of 5 anti-ANXA3 monoclonal antibodies amongst the 8 investigated. The epitopes determined from the comparison of the recognised overlapping peptide sequences are summarised in Table 11. The minimal epitope is the minimal sequence required to have recognition of the antibody, with a more or less intense signal. The optimal epitope is the ideal sequence allowing the best possible recognition of the antibodies, including or identical to the minimal epitope. The results obtained confirmed that TGC42 and TGC43 are well directed against a single epitope, the one described initially in application WO 2010/034825. Unexpectedly, the antibody 5C5B10 defines a new epitope which was not described in the prior art. The antibodies 6D9D10 and 9C6D4 are specific to the N-terminus end of the protein, like the monoclonal TGC7 of Patent Application WO 2007/141043. The monoclonal antibodies TGC44, 13A12G4H2 and 1F10A6 do not have any Spotscan reactivity, even on a membrane carrying peptides of 20 amino acids long. They probably possess conformational or at least semi-conformational epitopes of which the structures are not reproduced well enough by the synthetic peptides.

TABLE 11

| Anti- | Do- | Optimal | Minimal |
|---|---|---|---|
| body | main | epitope ^{a} | epitope ^{b} |
| TGC42 | D1 | SNAQRQLIVK | LIVKEYQAAYG |
| | | EYQAAYG | (55-65) |
| | | (49-65) | (SEQ ID |
| | | (SEQ ID | NO: 11) |
| | | NO: 10) | |
| TGC43 | D1 | IVKEYQAAYGKE | KEYQAAYG |
| | | (56-67) | (58-65) |
| | | (SEQ ID | (SEQ ID |
| | | NO: 12) | NO: 13) |
| 5C5B10 | D1 | DLSGHFEHL | LSGHFEH |
| | | (75-83) | (76-82) |
| | | (SEQ ID | (SEQ ID |
| | | NO: 14) | NO: 15) |
| 6D9D10 | N- | ASIWVGHRGTV | SIWVGHRGTV |
| | term | RDYPDFSPS | RDYPDFSP |
| | | (2-21) | (3-20) |
| | | (SEQ ID | (SEQ ID |
| | | NO: 16) | NO: 17) |
| 9C6D4 | N- | YPDF | YPDF |
| | term | (15-18) | (15-18) |
| | | (SEQ ID | SEQ ID |
| | | NO: 18) | NO: 18) |

^{a} Optimal epitope: Ideal sequence allowing the
best possible recognition of the antibody
(including or identical to the minimal epitope).
^{b} Minimal epitope: Minimal sequence required to
have a recognition of the antibody (more or less
intense signal).

Precise Localisation of the Epitope of the TGC44 Antibody by the Novatope Technique

The Novatope technique (Merck, Cat No. 69279) is a technology for analysing a protein in order to select the domains containing the epitopes. The method is based on the creation of a bank of bacterial clones, each expressing a fragment of the protein, cut randomly. These clones are analysed by immunovisualisation with the antibody that is to be characterised. The DNA sequencing of the positive clones serves to determine the protein sequence of a fragment containing the epitope. The technique was applied following the procedure supplied with the kit.

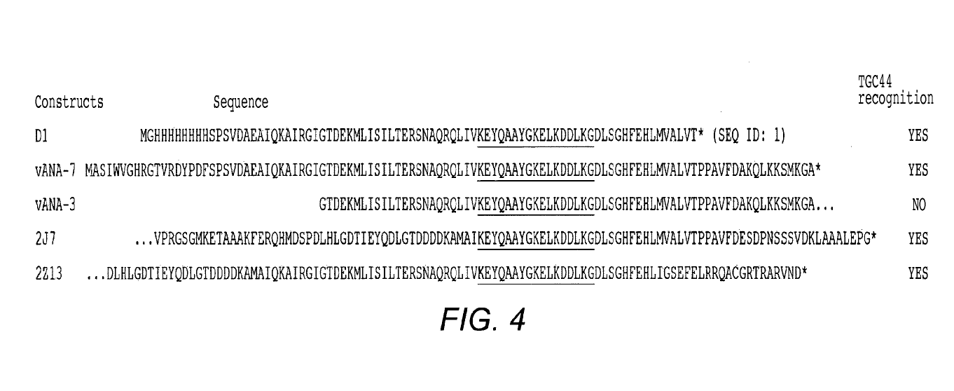

Thus, two clones reacting with the antibody TGC44 were able to be isolated and sequenced. Clone 2J7 expresses the KEYQAAYGKELKDDLKGDLSGHFEHLMVALVTPPAVFD sequence (SEQ ID NO: 19) which corresponds to the 58-95 residues of ANXA3. Clone 2Z13 expresses the QKAIRGIGTDEKMLISILTERSNAQRQLIVKEYQAAYGKELKDDLKGDLSGHFEHL sequence (SEQ ID NO: 20) which corresponds to the 28-83 residues of ANXA3. The common portion between the sequences of these two clones are the 58-83 residues of Annexin A3, that is the KEYQAAYGKELKDDLKGDLSGHFEHL sequence (SEQ ID NO: 21). Moreover, since the antibodies TGC44 and 5C5B10 are complementary (see Example 1), their two epitopes cannot overlap, so that the amino acids corresponding to the epitope of the 5C5B10 antibody, that is to say DLSGHFEHL (75-83) (SEQ ID: 14), can be withdrawn. Hence, the epitope of the TGC44 antibody is included in the KEYQAAYGKELKDDLKG (58-74) sequence (SEQ ID NO: 22). This is the same region as the one recognised by TGC42 and TGC43. On the other hand, the fact that the antibody TGC44 does not display Spotscan reactivity indicates a conformational limitation for recognition. TGC44 is capable of binding to its epitope only when the KEYQAAYGKELKDDLKG (58-74) sequence (SEQ ID NO: 22) is fused on the N-terminus side to a sequence with a length of at least 30 residues. Thus, the recombinant repeat domain D1, whose sequence is identified as SEQ ID NO: 6, the clones 2J7 and 2Z13 fused with a carrier protein or even the recombinant fragment vANA-7 described in application WO 2010/034825, are all recognised by the TGC44 clone. Conversely, the recombinant fragment vANA-3, which lacks the first 34 N-terminus amino acids, is not recognised (FIG. 4).

Precise Localisation of the Epitope of the 13A12G4H2 and 1F10A6 Antibodies

The experiment illustrated in FIG. 3 shows that the epitopes of the 13A12G4H2 and 1F10A6 antibodies are contained in domain D4 of Annexin A3. By directed mutagenesis, we constructed 12 recombinant proteins in order to improve the mapping of the antibodies directed against domain D4. This is the complete sequence of mature native ANXA3 (aa 2-323), fused on the N-terminus side to a histidine tag (non-mutated sequence). Mutagenesis by PCR was used to introduce alanine mutations at positions 253, 257, 260, 263, 265, 268, 270, 274, 306, 311 and 317 of the protein sequence (GeneArt Mutagenesis Service, Invitrogen). All these DNA fragments were cloned in the pMRCH79 vector (which is derived from the pMR78 vector), and then converted in bacteria BL21 (DE3). The proteins were produced and purified by techniques well known to a person skilled in the art and which have been mentioned at the beginning of this example.

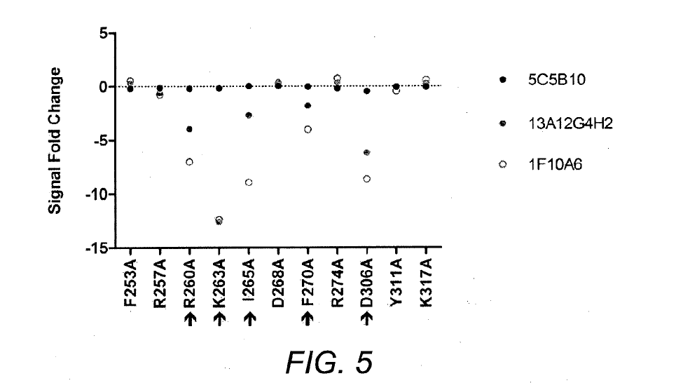

These 12 proteins (11 mutants and 1 non-mutated control) were then used to evaluate the binding capacity of the antibodies 13A12G4H2 and 1F10A6 in an ELISA in sandwich format, using the antibody TGC44 in capture. The detection antibody 5C5B10, whose epitope is in domain D1 and accordingly should not be affected by the mutations of domain D4, is used as control. The results are shown in FIG. 5. The graph shows the signal fold change (on the x-axis) of each mutation (on the y-axis). The signal fold change corresponds to the log₂ (mutated protein signal/non-mutated protein signal). The higher the absolute value of the signal fold change, the more the mutations for which this variation is observed affect the binding of the antibody. Thus, for 5C5B10, the “signal fold change” is always around 0, indicating that none of the mutations tested disturbs the binding of this monoclonal. On the other hand, for 13A12G4H2 and 1F10A6, it was possible to identify amino acids whose mutation prevents or disturbs the binding very significantly. These are the positions 260, 263, 265 and 306. The mutation of position 270 has a measurable but lesser impact than for preceding positions. We can therefore conclude that amino acids 260, 263, 265 and 306 of ANXA3 belong to the epitope recognised by the monoclonal antibodies 13A12G4H2 and 1F10A6, the two most important residues in the antigen-antibody interaction being lysine at position 263 and aspartic acid at position 306.

Determination of the Essential Amino Acids on the First Repeat Domain of Human Annexin A3 for the Recognition by the 5C5B10 and TGC43 Antibodies

For the purposes of determination of the essential amino acids of the epitopes included in D1 for the recognition by the 5C5B10 and TGC43 antibodies, the technique called “ALAscan” has been used presently. This “ALAscan” technique consists, during a peptide synthesis, in successively replacing the amino acids contained in an epitope by an alanine residue. This technique thus makes it possible to determine the essential amino acids for the recognition of the epitope by a given antibody. When the amino acid is strictly necessary for the interaction with the antibody, its replacement by an alanine causes the immunoreactivity of the peptide to disappear. When the amino acid is less important with regard to said interaction with the antibody, the immunoreactivity decreases slightly or remains unchanged. This technique was described by Geysen et al. in 1987 and can be broadened by replacing each amino acid by the 19 others.

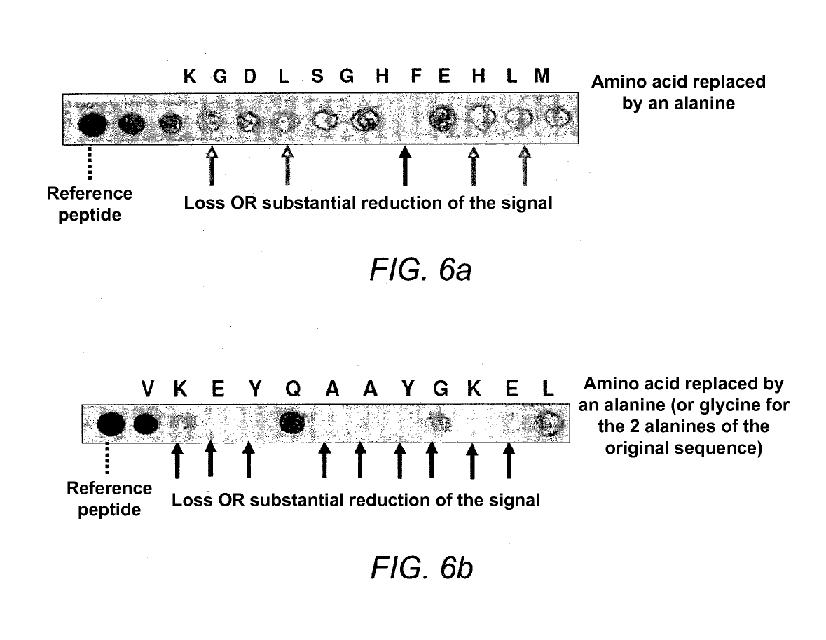

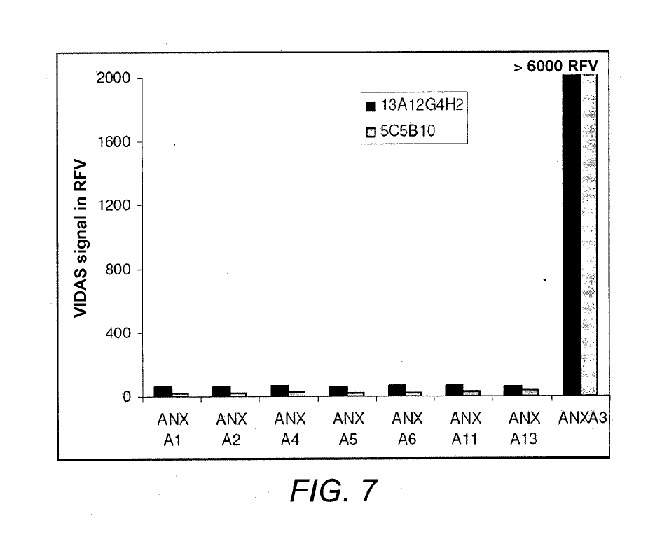

Thus, in order to determine the essential amino acids for the recognition of the aforesaid 5C5B10 antibody, the optimal consensus sequence of the epitope, such as shown in Table 11 above (SEQ ID NO: 14), “extended” by one or two amino acids on either side of this sequence, was synthesised on membrane by successively replacing each amino acid by an alanine. The reference peptide used is KGDLSGHFEHLM (residues 73 to 84 of human Annexin A3). The result obtained following the immunovisualisation of this membrane by the antibody 5C5B10 is presented in FIG. 6a.

This analysis shows that the primordial residue for the recognition of the epitope DLSGHFEHL by the 5C5B10 antibody is phenylalanine (F) in position 54 of SEQ ID NO: 1. There is in effect a total loss of the recognition when it is replaced by an alanine. The other residues which make an important contribution to the recognition are aspartic acid (D), serine (S), histidine (H) and leucine (L), respectively in positions 49, 51, 56 and 57 of SEQ ID NO: 1. It is important to recall that other mutations, namely the replacement of an amino acid by an analogous amino acid, will have little or no impact on the antigenic reactivity. Thus, the replacement of the glutamic acid by the aspartic acid should in principle not influence the attachment of the 5C5B10 antibody onto its epitope included in the first repeat domain (D1) of Annexin A3.

As indicated above, the expression “analogous amino acid” refers to an amino acid which, when it replaces the amino acid present in the epitope under investigation, does not cause any destruction of the antigenic reactivity of said epitope in respect of the antibody under investigation.

The particularly preferred analogues include naturally conservative substitutions, i.e. substitutions which take place in a family of amino acids. There are several family classifications of amino acids, as is well known to the person skilled in the art. Thus, according to one classification example, the amino acids can be divided into four families, namely (1) the acidic amino acids such as aspartic acid or glutamic acid, (2) the basic amino acids such as lysine, arginine and histidine, (3) non-polar amino acids such as leucine, isoleucine and methionine and (4) the non-charged polar amino acids such as glycine, asparagine, glutamine, serine, threonine and tyrosine. Phenylalanine, tryptophan and tyrosine are sometimes classed as aromatic amino acids. For example, it is possible to reasonably predict that an isolated replacement of a leucine residue by an isoleucine or valine residue, of an aspartate residue by a glutamate residue, of a threonine residue by a serine residue, or a similar conservative replacement of an amino acid by another amino acid which has a structural relationship, will have no major effect on the biological activity. Another example of a method which makes it possible to predict the effect of an amino acid substitution on the biological activity has been described by Ng and Henikoff, 2001

In the same way, the essential amino acids for the recognition of the antibody TGC43 have been determined using the reference peptide VKEYQAAYGKEL (residues 57-68 of Annexin A3). The two alanines contained in this sequence have been replaced by glycines during the ALAScan synthesis. The result obtained following the immunovisualisation of the ALAScan membrane by the antibody TGC43 is presented in FIG. 6b.

This analysis shows that 9 out of 10 residues are indispensable for the recognition of the epitope KEYQAAYGKE. In effect, there is a total or very significant loss of the recognition when each residue of the sequence is replaced by an alanine (or a glycine, if applicable), with the exception of glutamine (Q) in position 35 of SEQ ID NO: 1. As mentioned previously, it is important to recall that other mutations, namely the replacement of an amino acid by an analogous amino acid will have little or no impact on the antigenic reactivity. Thus the replacement of lysine residues by arginine residues, or glutamic acid by aspartic acid should not influence the attachment of the antibody TGC43.

### Example 8

### Analytical Specificity of ELISA Anti-ANXA3 Assay Formats

Annexins are a family of proteins which share homologies of function and of sequence. A BLAST interrogation made on the UniProtKB database, reduced to the sequences of human origin, served to identify the proteins having the greatest sequence homology with Annexin A3. These are, by decreasing order of homology, Annexin A4, Annexin A11, Annexin A6 and Annexin A5.

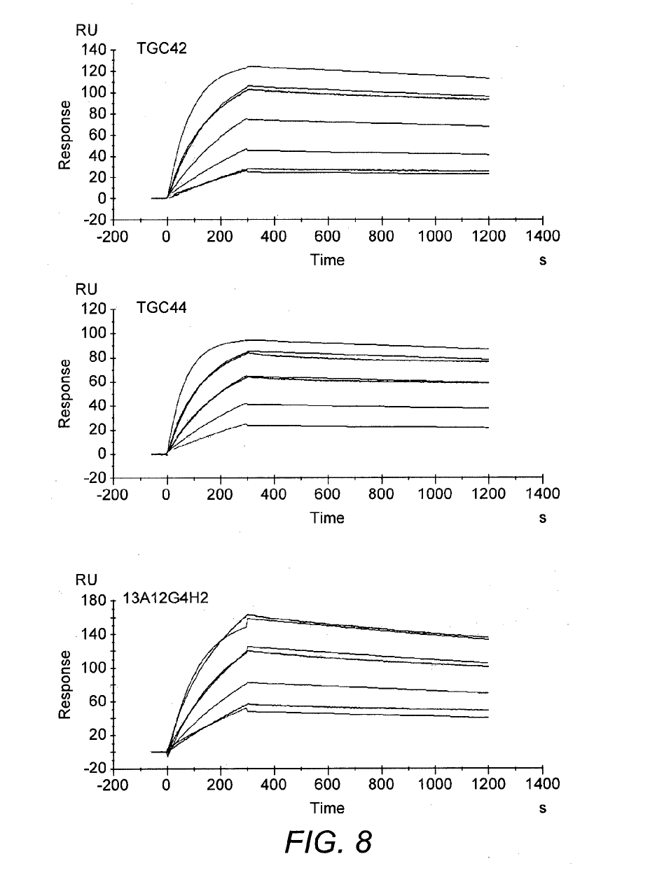

In consequence, it was important to demonstrate the specificity of the ELISA assays with regard to Annexin A3 and the absence of a cross-reaction with the other members of the family. Since the 3 antibodies TGC42, TGC43 and TGC44 are directed against the same epitope, the TGC44/13A12G4H2 assay (only referred to as 13A12G4H2 below) was selected as the prototype group 1 assay defined in Example 1. Similarly, the TGC44/5C5B10 assay (only referred to as 5C5B10 below) was selected as the prototype assay representing group 2. The absence of cross-reactivity was tested using commercially available antigens obtained from Abnova™: Annexin A1 (Cat. No. H00000301-P01), Annexin A2 (Cat. No. H00000302-P01), Annexin A4 (Cat. No. H00000307-P01), Annexin A5 (Cat. No. H00000308-P01), Annexin A6 (Cat. No. H00000309-P01) and Annexin A11 (Cat. No. H00000311-P01). Annexin A13 was expressed in recombinant form in the laboratory by cloning in the expression vector pMRCH79; fused to a histidine tag, and then purified by metal-chelate affinity chromatography. The proteins were diluted in the VIDAS® well X1 buffer to a concentration of 12.5 μg/mL for ELISA TGC44/5C5B10 and to a concentration of 16 μg/mL for ELISA TGC44/13A12G4H2. The results are shown in FIG. 7. The two ELISA formats TGC44/13A12G4H2 and TGC44/5C5B10 are both very specific to Annexin A3 and do not display cross-reactivity with the other Annexins tested.

### Example 9

### Determination of the Affinity of the Anti-Annexin A3 Antibodies

The surface plasmon resonance technology allows the real-time visualisation of the interactions between various unmarked biomolecules. One of the reagents is bound specifically to a biosensor (sensor chip) while the other species involved in the interaction is in a continuous flux of buffer. The surface plasmon resonance measurements were taken using a Biacore T100. The reagents including the sensor chip CM5, the mouse anti-IgG, specific to the fragment Fc, obtained in the rabbit (RAM Fc), and the kit for coupling via the amines for immobilising the antibodies were all obtained from GE-Healthcare Bioscience AB.

To investigate the kinetic fixing properties of the anti-Annexin A3 antibodies, the latter were immobilised by capture on the sensor chip, on which the RAM Fc antibody had previously been coupled covalently. The binding experiments were performed in buffer, HEPES, at 25° C., with a flow rate of 30 μL/min. The change in the resonance signal in RU (Resonance Units) allows the real-time monitoring of the binding and the dissociation of the biomolecules on the surface of the sensor chip. In a first step, the monoclonal antibody to be investigated was injected into channel 2 to obtain a signal of about 250 RU. Then, the Annexin A3 (Arodia) was injected into channels 1 and 2. The association and dissociation times were 5 and 15 minutes, respectively. After measuring the resonance responses, the surface of the sensor chip was regenerated by washing with 50 mM HCl, at 10 μL/min for 120 seconds. The same measurement method was repeated for each dilution of the Annexin A3 protein; a total of 9 different dilutions of the protein, between 0 and 64 nM, were analysed. The sensorgrams obtained were plotted and analysed with the dedicated Biacore T100 software, using the 1:1 interaction model. The kinetic association (Kon) and dissociation (Koff) constants were measured using the antibodies in a concentration of 3 μg/mL, except for 13A12G4H2 and 1F10A6, which were used at 0.75 μg/mL in order to limit the impact of the background noise. The affinity, represented by the dissociation constant (Kd), was calculated (Kd=Koff/Kon).

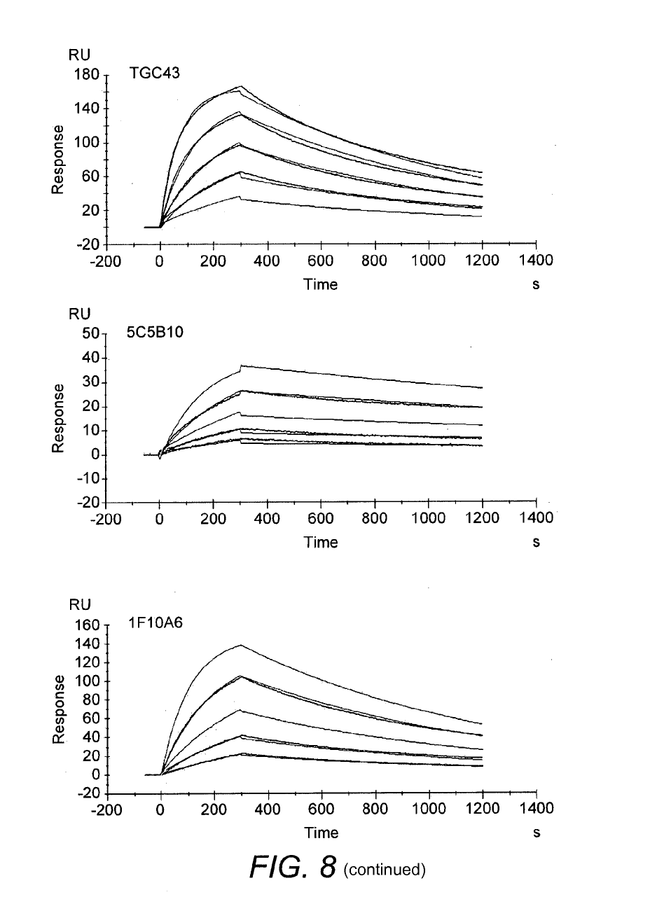

For each anti-Annexin A3 antibody, the graphs showing the resonance signal as a function of time are shown in FIG. 8. The measured values of the association and dissociation constants, and the calculated value of the affinity constants, are given in Table 12.

All the anti-ANXA3 antibodies investigated had high affinities, with Kd ranging from 10⁻⁹ to 5×10⁻¹⁰ M. It is possible to class the antibodies into two distinct groups according to their Kd. The first group corresponds to the antibodies 13A12G4H2, TGC42 and TGC44 of which the Kd is higher than 10⁻¹⁰ M, which is the group of very high affinity antibodies. The second group corresponds to the antibodies 5C5B10, 1F10A6 and TGC43 of which the Kd is between 10⁻⁹ and 3.5×10⁻⁹ M, which is the group of high affinity antibodies.

The three antibodies used in capture, TGC42, TGC43 and TGC44, have equivalent kinetic association constants. The antibody 13A12G4H2 also has a comparable kinetic association constant. The kinetic association constant of the antibody 5C5B10 is about 1 log lower than that of TGC44.

With regard to the kinetic dissociation constants, it is also possible to divide the anti-ANXA3 antibodies into 2 groups. The first group contains TGC44, TGC42, 13A12G4H2 and 5C5B10; it is characterised by a very low kinetic dissociation constant, between 9×10⁻⁵ and 3.5×10⁻⁴ s⁻¹. Once the antigen-antibody fixing has taken place, the Annexin A3 is retained by the antibodies of this group and does not dissociate. The second group contains TGC43 and 1F10A6; it is characterised by a higher kinetic dissociation constant of the order of 10⁻³ s⁻¹. The antibodies of this group, even if they succeed in fixing the Annexin A3, dissociate much faster. Thus, although they have comparable kinetic association constants, TGC42 and TGC44 are better capture antibodies to be used for developing an ELISA assay than TGC43.

TABLE 12

| Antibody bound to | | | |
|---|---|---|---|
| the sensor chip | Kon (M⁻¹ · s⁻¹) | Koff (s⁻¹) | Kd (M) |
| 13A12G4H2 | 5.3E+05 | 2.7E−04 | 5.0E−10 |
| TGC42 | 7.3E+05 | 1.0E−04 | 1.8E−10 |
| TGC44 | 9.5E+05 | 9.7E−05 | 1.0E−10 |
| 5C5B10 | 9.8E+04 | 3.4E−04 | 3.4E−09 |
| 1F10A6 | 3.8E+05 | 1.1E−03 | 2.9E−09 |
| TGC43 | 8.4E+05 | 1.1E−03 | 1.3E−09 |

### Example 10

### Presence of ANXA3 in Human Beings

Exosomes are membranal vesicles having a diameter of 40-100 nm secreted by various cell types in vivo. They are found in the blood, urine, the malignant ascites liquids and contain nucleic and protein markers of tumoral cells, from which they are secreted.

In order to detect the presence of serum exosomal ANXA3, an exosome enrichment from the serum was performed for 2 patients A and B suffering from prostate tumours, using the ExoQuick exosome precipitation solution (System Biosciences, Cat. No. EXOQ20A-1), following the manufacturer's instructions. For each serum sample A and B, the proteins isolated by exosomal enrichment were analysed by migration on 10% SDS-PAGE gel. After resuspension of the exosomal residue in RIPA buffer (G-Biosciences, Cat. No. 786-489), a test sample corresponding to 1/10^{th} of the solution is heated for 4 min at 95° C. in the presence of 5× Laemmli load buffer and then deposited on gel in reducing conditions. The Precision Plus Protein molecular weight marker (Bio-Rad) is simultaneously deposited on gel as a molecular weight control. In order to analyse the specific presence of exosomal ANXA3, a Western blot is performed following a standard protocol. The SDS-PAGE gel is transferred to PVDF membrane (iblot Gel Transfer Stacks PVDF, Cat. No. IB4010-02), the membrane is stained for 1 min in amino-black solution and then rinsed with distilled water.

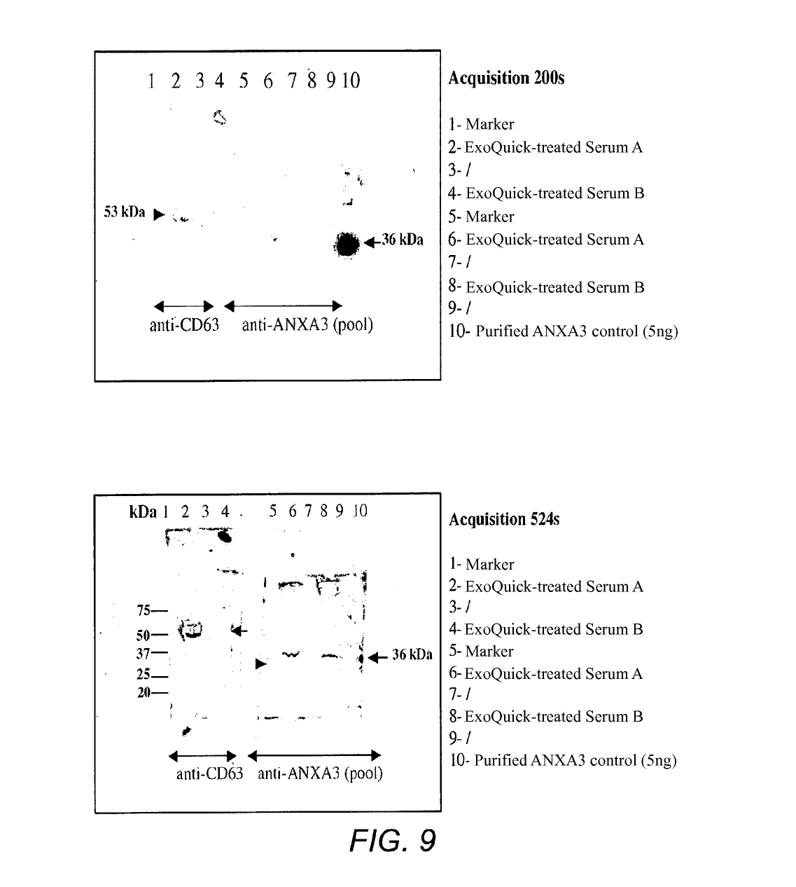

For the study, an anti-CD63 monoclonal antibody (Santa Cruz, Cat. No. SC-5275) directed against the common exosomal protein marker Tetraspanine CD63, and a pool of 3 anti-ANXA3 monoclonal antibodies (TGC44, 13A12G4H2, 5C5B10) were used in detection. The immune reaction is performed by incubation of the membrane overnight at +4° C. with slow stirring in the presence of the primary antibodies diluted in 5% TNT-milk buffer in the concentrations of 4 μg/mL for anti-CD63 and 10 μg/mL for the pool of anti-ANXA3 antibodies. The immune complexes are developed by AffiniPure mouse anti-IgG goat secondary antibody combined with peroxidase (H+L, Jackson Immunoresearch Cat. No. 115-035-062), diluted to 1/5000^{th} in 5% TNT-milk and incubated for 1 hour at ambient temperature. The signals developed by “enhanced chemiluminescence” (ECL, Supersignal West substrate Pierce) are visualised on the Molecular Imager ChemiDoc XRS+ platform (BioRad) with the Quantity One 4.6.9 software. The results are given in FIG. 9. They show that it is possible to detect Annexin A3 in the exosomes precipitated by ExoQuick from the serum of patients.

It is also possible to assay Annexin A3 after purification of the exosomes. For this purpose, an adjustment of the sandwich assay developed on VIDAS® using the TGC44 antibody in capture and the biotinylated 13A12G4H2 antibody in detection was made in Luminex format. The technique uses microbeads in suspension to detect and quantify several biomolecules in the same small-volume sample with high sensitivity.

Magnetic beads 5.6 μm in diameter, having a spectral address based on their red/infrared content, served as the support for the assay. A quantity of 9 μg of TGC44 capture antibody was grafted onto the surface of the magnetic beads (Bio-Rad, Bio-Plex Pro Magnetic COOH Beads Amine Coupling Kit) following the manufacturer's instructions.

Annexin A3 was assayed in the serum of 4 patients suffering from a prostate tumour, before and after exosomal enrichment (ExoQuick precipitation solution). To assay the serum, the samples B, C, D and E are diluted to ⅕^{th} in TBST buffer. The assay of the Annexin A3 after treatment of the serum and purification of the exosomes is performed with ⅓ of the exosomal residue taken up in distilled water qsp 100 μL. The samples are incubated in the presence of 5000 beads coupled with the capture antibody in a 96 well plate (Bio-Rad, Cat No. 171025001) for 2 hours at 37° C., 650 rpm, sheltered from light. Between each step, the wells are washed 3 times in 0.05% TBST. Detection is performed with 100 μL of biotinylated 13A12G4H2 secondary antibody in a concentration of 0.005 μg/mL for 1 hour at 37° C. with stirring. Visualisation of the immune complex takes place by incubation of 100 μL of streptavidin solution coupled with phycoerythrine (RPE) in a concentration of 2 μg/mL (Dako) for 30 minutes at 37° C. with stirring. The final step consists in resuspending the immune complexes in 100 μL of TBS for a flux fluorimetry analysis performed by the Bio-Plex 200 robot (Bio-Rad). Each bead undergoes a double excitation by a red laser (633 nm) for its identification and a green laser (532 nm) for quantification of the analyte by measurement of the fluorescent conjugate.

The results of the direct assay in the serum and after exosome enrichment by ExoQuick treatment are given in Table 13 below. This experiment clearly shows that it is possible to assay Annexin A3 directly in exosomes purified from serum by the inventive method.

TABLE 13

| | ANXA3 assay in ng/mL | |
|---|---|---|
| | | Assay after ExoQuick treatment |
| Serum | Direct assay of serum | (enrichment) |
| D | 4.38 | 27.98 |
| E | 4.78 | 23.25 |
| B | 5.62 | 53.58 |
| C | 17.98 | 148.28 |

### Example 11

### Antigenic Reactivity Expected Between the Homologous Protein Sequences of Epitopes in Various Mammals and Certain Human Anti-ANXA3 Antibodies

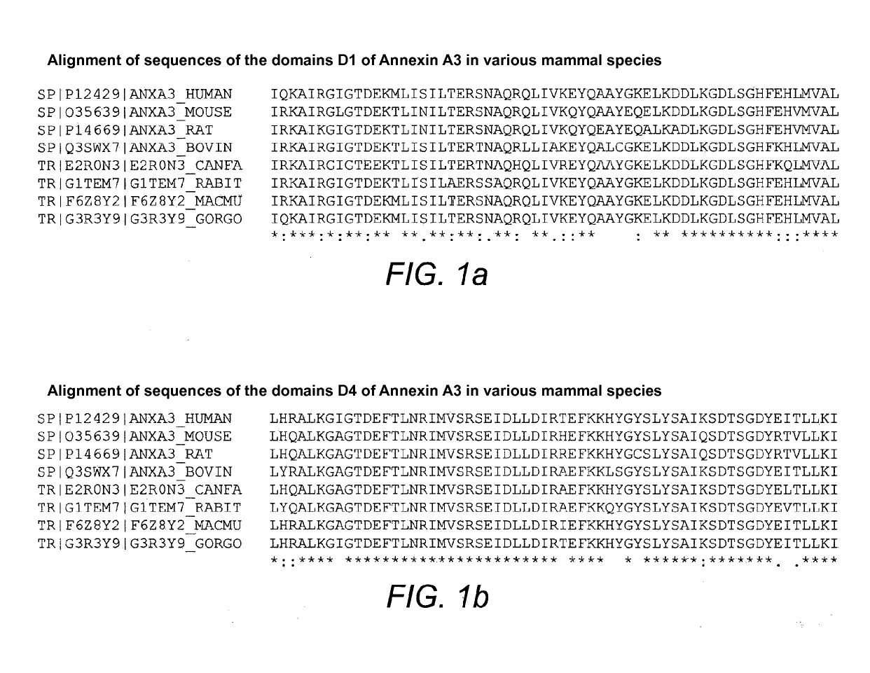

As shown in Table 1 and depicted in FIGS. 1a and 1b, the sequences of the first and fourth repeat domains (D1 and D4 respectively) of Annexin A3 are well conserved amongst the main mammals and demonstrate a large identity percentage. As a consequence of this, the applicants have sought to predict the antigenic reactivity between the homologues of the epitopes LIVKEYQAAYGKE and DLSGHFEHL in various mammals and the antibodies TGC42, TGC43 and TGC44 on the one hand and 5C5B10 on the other hand.

As is known to the person skilled in the art, certain amino acid substitutions can influence, i.e. decrease or increase the antigenic reactivity of an epitope relative to a given antibody, whereas the substitution by another amino acid in the same position will have no influence. The classification of the amino acids mentioned in the description, as well as the substitution matrices, such as that by Gonnet for example make it possible to predict with reasonable chances of success the impact which a given mutation can have at a precise place in an epitope.

The experimental ALAScan data presented in Example 7 have made it possible to identify the amino acids directly implicated in the epitope-antibody interaction for each of the epitopes LIVKEYQAAYGKE and DLSGHFEHL. In the same way, the PCR mutagenesis data presented in the same example have made it possible to determine the residues which are strictly necessary for the recognition of the epitope by the monoclonal antibodies 13A12G4H2 and 1F10A6, an epitope which also proves to be the primary Ca2+ attachment site of the domain D4 of Annexin A3. From these experimental data and relying on the teaching of substitution matrices (such as Gonnet's), it is possible to predict the antigenic reactivity between the homologues of the epitopes LIVKEYQAAYGKE and DLSGHFEHL in the Annexins A3 of other mammals and the corresponding antibodies which attach to the first repeat domain of human Annexin A3. These analyses are presented hereafter, in Tables 14 and 15 respectively.

TABLE 14
Sequence of the epitope LIVKEYQAAYGKE
of Annexin A3 in other mammals and
recognition the antibodies expected
by TGC42, TGC43 and TGC44

| | | Ex- | |
|---|---|---|---|
| | | pected | |
| | | reac- | |
| Identifier | Sequence | tivity | Comments |
| SP\|P12429\| | LIVKEYQA | + | |
| ANXA3_HUMAN | AYGKE | | |
| SP\|O35639\| | LIVKQYQA | - | |
| ANXA3_MOUSE | AYEQE | | |
| SP\|P14669\| | LIVKQYQE | - | |
| ANXA3_RAT | AYEQA | | |
| SP\|Q3SWX7\| | LIAKEYQA | +/- | |
| ANXA3_BOVIN | LCGKE | | |
| TR\|E2R0N3\| | LIVREYQA | + | K and R are |
| E2R0N3_CANFA | AYGKE | | analogous |
| TR\|G1TEM7\| | LIVREYQA | + | K and R are |
| G1TEM7_RABIT | AYGKE | | analogous |
| TR\|F6Z8Y2\| | LIVKEYQA | + | |
| F6Z8Y2_MACMU | AYGKE | | |
| TR\|G3R3Y9\| | LIVKEYQA | + | |
| G3R3Y9_GORGO | AYGKE | | |

TABLE 15
Sequence of the epitope DLSGHFEHL
of Annexin A3 in other mammals
and recognition expected
by the antibody 5C5B10

| | | Ex- | |
|---|---|---|---|
| | | pected | |
| | | reac- | |
| Identifier | Sequence | tivity | Comments |
| SP\|P12429\| | DLSGHFEHL | + | |
| ANXA3_HUMAN | | | |
| SP\|O35639\| | DLSGHFEHV | + | L and V are |
| ANXA3_MOUSE | | | analogous |
| SP\|P14669\| | DLSGHFEHV | + | L and V are |
| ANXA3_RAT | | | analogous |
| SP\|Q3SWX7\| | DLSGHFKHL | + | K replaces |
| ANXA3_BOVIN | | | the E which |
| | | | is not |
| | | | involved in |
| | | | the binding |
| TR\|E2R0N31 | DLSGHFKQL | +/- | |
| E2R0N3_CANFA | | | |
| TR\|G1TEM7\| | DLSGHFEHL | + | |
| G1TEM7_RABIT | | | |
| TR\|F6Z8Y21 | DLSGHFEHL | + | |
| F6Z8Y2_MACMU | | | |
| TR\|G3R3Y9\| | DLSGHFEHL | + | |
| G3R3Y9_GORGO | | | |

With regard to the epitope which corresponds to the Ca2+ ion binding site in the domain D4 (epitope recognised by the monoclonal antibodies 13A12G4H2 and 1F10A6), mutagenesis by PCR (cf. Example 7) made it possible to show that lysine (K) at position 263 was strictly necessary to the epitope-antibody interaction. This residue is conserved in all of the sequences presented in 1b, it is found at position 6 of the amino acid sequence of the fourth repeat domain of Annexin A3 (domain D4). The second most important residue is aspartic acid (D) at position 306 of Annexin A3. It is also conserved in all of the sequences presented in FIG. 1b and is found at position 49 of the amino acid sequence of the fourth repeat domain of Annexin A3 (domain D4). The conservation of these two positions makes it possible to predict that the antibodies 13A12G4H2 and 1F10A6 would be able to attach to all of the mammalian Annexins A3 presented in this alignment. However, arginine (R) at position 260 and isoleucine (I) at position 265 are not always conserved. The data regarding mutagenesis by PCR show that these positions are also implicated in the recognition, but to a lesser degree (cf. Example 7). The antibody attachment signal decreases when these positions are replaced by an alanine but the attachment is not totally eradicated. It will be possible to compensate for this lowering of the signal by means of an increase in the use concentration of the detection antibodies. The antibodies divulged possess a very great affinity for human Annexin A3 and, as a result, are used in quite a diluted manner in the assays of the invention. For example, the antibody 13A12G4H2 can be used at a concentration in the 1-20 pg/mL range (concentration in the conjugated buffer X1 which is also the sample's dilution buffer, dilution=300 μL conjugated buffer+150 μL biological sample). To be able to assay the mammalian Annexins A3 bearing mutations at positions 260 and 265, the person skilled in the art shall easily be able to determine a new optimal antibody use concentration which can be 10, 100 or even 1000 times greater than when the target of the assay is human Annexin A3 while still remaining lower than a concentration of approximately 30 μg/mL in the conjugated buffer X1.

### Example 12

### Reproducibility of the Assays TGC44/13A12G4H2 and TGC44/5C5B10

The objective of the experiment presented in this example is to study an important analytical characteristic of the assays ELISA TGC44/13A12G4H2 and TGC44/5C5B10, namely the reproducibility. This analyse was achieved by using biological samples, i.e. of serum or urine collected just after the rectal touch (post-rectal touch urine also referred to as “expressed urine”) obtained in patients suffering from prostate pathologies (prostate cancer or benign prostatic hyperplasia), in order to get as close as possible to the real use conditions of the assays. The reproducibility of the assay TGC44/5C5B10 on the serum samples has not been studied because this assay does not allow the assaying of human Annexin A3 in the serum.

For each assay and each type of sample, 5 samples were assayed on 1 instrument in 3 days, at a rate of 2 series per day and 2 repetitions per series. There are thus n=12 measuring repetitions for each sample. The coefficient of variation (CV) corresponding to this series of 12 measurements was calculated and is presented in Table 16 below. This coefficient of variation is the indicator which makes it possible to judge the reproducibility of the measurement: the smaller the CV, the more reproducible the measurement.

This analysis makes it necessary to have large volumes of samples in order to be able to carry out repeated measurements. Compared to urine, it is less easy to obtain large volumes of serum. Thus the experiments with urine have been carried out on individual urines whereas certain serums have been “pooled”, to obtain the necessary volume.

TABLE 16
Reproducibility of the ELISA TGC44/13A12G4H2 and TGC44/5C5B10

| TGC44/ | | | | | |
|---|---|---|---|---|---|
| 13A12G4H2 | | | | TGC44/5C5B10 | |
| On post-rectal | | TGC44/13A12G4H2 | | On post-rectal touch | |
| touch urine | | on serum | | urine | |
| Sample | % CV | Sample | % CV | Sample | % CV |
| 50237 | 8 | L02 + L13 | 4 | 50222 | 15 |
| 10204 | 11 | L04 + L07 | 6 | 10204 | 9 |
| 30299 | 5 | L01 | 6 | 50237 | 13 |
| 1001450 | 7 | L41 | 4 | 1001450 | 14 |
| 1001425 | 7 | N03 + N05 + N11 | 4 | 1001425 | 11 |

The ELISA TGC44/13A12G4H2 possesses good reproducibility when it is used to assay post-rectal touch urines. In effect, for the 5 samples tested, the CV are lower than 15%, which represents the admitted upper limit. ELISA TGC44/13A12G4H2 also possesses very good reproducibility to assay serums, better than in urine. Amongst the serum samples tested, none exceeds a CV of 6%. The assay TGC44/5C5B10 also possesses a correct reproducibility in post-rectal touch urine, albeit a little less good than ELISA TGC44/13A12G4H2.

### REFERENCES

1. Moss S E, Morgan R O. The annexins. Genome biology 2004; 5: 219.
2. Barton G J, Newman R H, Freemont P S, Crumpton M J. Amino acid sequence analysis of the annexin super-gene family of proteins. Eur J Biochem 1991; 198: 749-760.
3. Gerke V, Moss S E. Annexins: from structure to function. Physiological reviews 2002; 82: 331-371.
4. Gerke V, Creutz C E, Moss S E. Annexins: linking Ca2+ signalling to membrane dynamics. Nature reviews. Molecular cell biology 2005; 6: 449-461.
5. Hayes M J, Rescher U, Gerke V, Moss S E. Annexin-actin interactions. Traffic 2004; 5: 571-576.
6. Rescher U, Gerke V. Annexins—unique membrane binding proteins with diverse functions. Journal of cell science 2004; 117: 2631-2639.
7. Harashima M, Niimi S, Koyanagi H et al. Change in annexin A3 expression by regulatory factors of hepatocyte growth in primary cultured rat hepatocytes. Biological & pharmaceutical bulletin 2006; 29: 1339-1343.
8. Le Cabec V, Russo-Marie F, Maridonneau-Parini I. Differential expression of two forms of annexin 3 in human neutrophils and monocytes and along their differentiation. Biochem Biophys Res Commun 1992; 189: 1471-1476.
9. Le Cabec V, Maridonneau-Parini I. Annexin 3 is associated with cytoplasmic granules in neutrophils and monocytes and translocates to the plasma membrane in activated cells. Biochem J 1994; 303 (Pt 2), 481-487.
10. Bianchi C, Bombelli S, Raimondo F et al. Primary cell cultures from human renal cortex and renal-cell carcinoma evidence a differential expression of two spliced isoforms of Annexin A3. Am J Pathol 176: 1660-1670.
11. Tait J F, Sakata M, McMullen B A et al. Placental anticoagulant proteins: isolation and comparative characterization of four members of the lipocortin family. Biochemistry 1988; 27: 6268-6276.
12. Ernst J D, Hoye E, Blackwood R A, Jaye D. Purification and characterization of an abundant cytosolic protein from human neutrophils that promotes Ca2 (+)-dependent aggregation of isolated specific granules. The Journal of clinical investigation 1990; 85: 1065-1071.
13. Chong K W, Chen M J, Koay E S et al. Annexin A3 is associated with cell death in lactacystin-mediated neuronal injury. Neuroscience letters 2010; 485: 129-133.
14. Kollermann J, Schlomm T, Bang H et al. Expression and prognostic relevance of annexin A3 in prostate cancer. Eur Urol 2008; 54: 1314-1323.
15. Wozny W, Schroer K, Schwall G P et al. Differential radioactive quantification of protein abundance ratios between benign and malignant prostate tissues: cancer association of annexin A3. Proteomics 2007; 7: 313-322.
16. Madoz-Gurpide J, Lopez-Serra P, Martinez-Torrecuadrada J L et al. Proteomics-based validation of genomic data: applications in colorectal cancer diagnosis. Molecular & cellular proteomics: MCP 2006; 5: 1471-1483.
17. Marshall K W, Mohr S, Khettabi F E et al. A blood-based biomarker panel for stratifying current risk for colorectal cancer. International journal of cancer. Journal international du cancer 2010; 126: 1177-1186.
18. Liu Y F, Xiao Z Q, Li M X et al. Quantitative proteome analysis reveals annexin A3 as a novel biomarker in lung adenocarcinoma. The Journal of pathology 2009; 217: 54-64.
19. Baine M J, Chakraborty S, Smith L M et al. Transcriptional profiling of peripheral blood mononuclear cells in pancreatic cancer patients identifies novel genes with potential diagnostic utility. PLoS One 2011; 6: e17014.
20. Jung E J, Moon H G, Park S T et al. Decreased annexin A3 expression correlates with tumor progression in papillary thyroid cancer. Proteomics. Clinical applications 2010; 4: 528-537.
21. Wang X M, Wu T X, Hamza M et al. Rofecoxib modulates multiple gene expression pathways in a clinical model of acute inflammatory pain. Pain 2007; 128: 136-147.
22. Kohler G, Milstein C. Continuous cultures of fused cells secreting antibody of predefined specificity. Nature 1975; 256: 495-497.
23. Kohler G, Howe S C, Milstein C. Fusion between immunoglobulin-secreting and nonsecreting myeloma cell lines. Eur J Immunol 1976; 6: 292-295.
24. Frank R, Daring R. Simultaneous multiple peptide under continuous flow conditions on cellulose paper discs as segmental solid synthesis. Tetrahedron 1988; 44: 6031-6040.
25. Gonnet G. H., Cohen M. A., Benner S. A.; “Exhaustive matching of the entire protein sequence database.”; Science 256:1443-1445 (1992)
26. Geysen M S, Rodda T, Mason T J, Triubbick G, Schoofs P. Strategies of epitope analysis using synthetic peptide synthesis. J. Immunol. Meth. 1987. 102: 259-265.
27. Ng and Henikoff, 2001, Genome Res 11: 863-874.

1. A method of in vitro detection and/or assaying of Annexin A3 in a biological sample from a mammal, said biological sample being chosen from amongst blood or at least one of its derivatives such as plasma and serum, wherein the biological sample is brought into contact with a first antibody and a second antibody, the first antibody being directed against the first repeat domain of Annexin A3 from said mammal and the second antibody being directed against the fourth repeat domain of Annexin A3 from said mammal.

2. The method according to claim 1, wherein the first antibody is a capture antibody and the second antibody is a detection antibody.

3. The method according to claim 1, wherein the first antibody is selected from amongst the antibodies directed against an epitope, of which the amino acid sequence comprises at least 7 consecutive amino acids and no more than 17 consecutive amino acids of the first repeat domain of Annexin A3 from said mammal.

4. The method according to claim 1, wherein the first antibody is selected from amongst the antibodies directed against an epitope included in the first repeat domain of Annexin A3 from said mammal, said epitope having an amino acid sequence selected from amongst the following sequences:
Xaa1-Xaa2-A-Q-Xaa3-Xaa4-L-I-Xaa5-Xaa6-Xaa7-Y-Q-Xaa8-Xaa9-Xaa10-Xaa11 (SEQ ID NO: 37),
L-I-Xaa5-Xaa6-Xaa7-Y-Q-Xaa8-Xaa9-Xaa10-Xaa11 (SEQ ID NO: 38),
I-Xaa5-Xaa6-Xaa7-Y-Q-Xaa8-Xaa9-Xaa10-Xaa11-Xaa12-Xaa13 (SEQ ID NO: 39),
Xaa6-Xaa7-Y-Q-Xaa8-Xaa9-Xaa10-Xaa11 (SEQ ID NO: 40),
Xaa6-Xaa7-Y-Q-Xaa8-Xaa9-Xaa10-Xaa11-Xaa12-Xaa13-L-K-Xaa14-D-L-K-G (SEQ ID NO: 41) provided that the amino acid sequence SEQ ID NO: 41 is fused on the N-terminus side to a sequence of at least 30 amino acids,
DLSGHF-Xaa20-Xaa21-Xaa22 (SEQ ID NO: 42),
LSGHF-Xaa20-Xaa21 (SEQ ID NO: 43),
wherein:
Xaa1 represents a serine residue, a threonine residue, or an analogous amino acid,
Xaa2 represents an asparagine residue, a serine residue or an analogous amino acid,
Xaa3 represents an arginine residue, a histidine residue or an analogous amino acid,
Xaa4 represents a glutamine residue, a leucine residue or an analogous amino acid,
Xaa5 represents a valine residue, an alanine residue or an analogous amino acid,
Xaa6 represents a lysine residue, an arginine residue or an analogous amino acid,
Xaa7 represents a glutamic acid residue, a glutamine residue or an analogous amino acid,
Xaa8 represents an alanine residue, a glutamic acid residue or an analogous amino acid,
Xaa9 represents an alanine residue, a leucine residue or an analogous amino acid,
Xaa10 represents a tyrosine residue, a cysteine residue or an analogous amino acid,
Xaa11 represents a glycine residue, a glutamic acid residue or an analogous amino acid,
Xaa1.2 represents a lysine residue, a glutamine residue or an analogous amino acid,
Xaa13 represents a glutamic acid residue, an alanine residue or an analogous amino acid,
Xaa14 represents an aspartic acid residue, an alanine residue or an analogous amino acid,
Xaa20 represents a glutamic acid residue, a lysine residue or an analogous amino acid,
Xaa21 represents a histidine residue, a glutamine residue or an analogous amino acid,
Xaa22 represents a leucine residue, a valine residue or an analogous amino acid.

5. The method according to claim 1, wherein the second antibody is selected from amongst the antibodies directed against an epitope, of which the amino acid sequence comprises at least 7 consecutive amino acids and no more than 50 consecutive amino acids of the fourth repeat domain of Annexin A3 from said mammal.

6. The method according to claim 1, wherein the second antibody is selected from amongst the antibodies directed against an epitope, of which the amino acid sequence corresponds to the amino acid sequence starting at residue 3 and ending at residue 49 of the fourth repeat domain of Annexin A3 from said mammal.

7. The method according to claim 5, wherein the epitope comprises a lysine residue at position 6 of the fourth repeat domain of Annexin A3 from said mammal and/or an aspartic acid residue at position 49 of said fourth repeat domain of Annexin A3 from said mammal.

8. The method according to claim 7, wherein the epitope comprises an arginine or glutamine residue at position 3 of the fourth repeat domain of Annexin A3 from said mammal and an isoleucine or alanine residue at position 8 of said fourth repeat domain of Annexin A3 from said mammal.

9. The method according to claim 7, wherein the epitope comprises a glycine residue at position 7 of the fourth repeat domain of Annexin A3 from said mammal, an isoleucine or alanine residue at position 8 of the fourth repeat domain of Annexin A3 from said mammal and a glycine residue at position 9 of said fourth repeat domain of Annexin A3 from said mammal.

10. The method according to claim 1, wherein the mammal is human and wherein the first antibody is directed against the first repeat domain of said human Annexin A3, the sequence of which is identified as SEQ ID NO: 1, and the second antibody is directed against the fourth repeat domain of human Annexin A3, the sequence of which is identified as SEQ ID NO: 2.

11. The method according to claim 1 for the in vitro diagnosis and/or the in vitro monitoring of the evolution of a pathology comprised in the group constituted by cancers, such as urogenital cancers and particularly prostate cancers, colorectal cancer, lung cancer, pancreatic cancer, papillary thyroid cancer, breast cancer, inflammatory disorders such as systemic inflammatory response syndrome, sepsis, rheumatoid polyarthritis, Crohn's disease, diabetes.

12. A kit for the immunodetection and/or immunoassay of a biological sample from a mammal, said biological sample being chosen from amongst blood or at least one of its derivatives such as plasma and serum, said kit comprising:
a first antibody directed against the first repeat domain of Annexin A3 from said mammal, as defined in claim 1,
a second antibody directed against the fourth repeat domain of Annexin A3 from said mammal, as defined in claim 1,
said immunoassay kit making it possible to implement the method according to claim 1.

13. The kit according to claim 12, said kit comprising an appropriate calibration means.

14. The use of a kit according to claim 12 for the implementation of a method of in vitro detection and/or assaying of Annexin A3 in a biological sample from a mammal, said biological sample being chosen from amongst blood or at least one of its derivatives such as plasma and serum, wherein the biological sample is brought into contact with a first antibody and a second antibody, the first antibody being directed against the first repeat domain of Annexin A3 from said mammal and the second antibody being directed against the fourth repeat domain of Annexin A3 from said mammal.


SEQUENCE LISTING (54 sequences)

SEQ ID NO: 1 - protein, 61 aa, Homo sapiens
IQKAIRGIGTDEKMLISILTERSNAQRQLIVKEYQAAYGKELKDDLKGDLSGHFEHLMVAL

SEQ ID NO: 2 - protein, 61 aa, Homo sapiens
LHRALKGIGTDEFTLNRIMVSRSEIDLLDIRTEFKKHYGYSLYSAIKSDTSGDYEITLLKI

SEQ ID NO: 3 - protein, 61 aa, Homo sapiens
LKKSMKGAGTNEDALIEILTTRTSRQMKDISQAYYTVYKKSLGDDISSETSGDFRKALLTL

SEQ ID NO: 4 - protein, 61 aa, Homo sapiens
YKAGENRWGTDEDKFTEILCLRSFPQLKLTFDEYRNISQKDIVDSIKGELSGHFEDLLLAI

SEQ ID NO: 5 - protein, 323 aa, Homo sapiens
MASIWVGHRGTVRDYPDFSPSVDAEAIQKAIRGIGTDEKMLISILTERSNAQRQLIVKEYQAAYGKELKDDLKGDLSGHFEHLMVALVTPPAVFDAKQLKKSMKGAGTNEDALIEILTTRTSRQMKDISQAYYTVYKKSLGDDISSETSGDFRKALLTLADGRRDESLKVDEHLAKQDAQILYKAGENRWGTDEDKFTEILCLRSFPQLKLTFDEYRNISQKDIVDSIKGELSGHFEDLLLAIVNCVRNTPAFLAERLHRALKGIGTDEFTLNRIMVSRSEIDLLDIRTEFKKHYGYSLYSAIKSDTSGDYEITLLKICGGDD

SEQ ID NO: 6 - protein, 81 aa, Artificial Sequence
  Recombinant D1
MGHHHHHHHHSPSVDAEAIQKAIRGIGTDEKMLISILTERSNAQRQLIVKEYQAAYGKELKDDLKGDLSGHFEHLMVALVT

SEQ ID NO: 7 - protein, 79 aa, Artificial Sequence
  Recombinant D2
MGHHHHHHHHAVFDAKQLKKSMKGAGTNEDALIEILTTRTSRQMKDISQAYYTVYKKSLGDDISSETSGDFRKALLTLA

SEQ ID NO: 8 - protein, 85 aa, Artificial Sequence
  Recombinant D3
MGHHHHHHHHDEHLAKQDAQILYKAGENRWGTDEDKFTEILCLRSFPQLKLTFDEYRNISQKDIVDSIKGELSGHFEDLLLAIVN

SEQ ID NO: 9 - protein, 82 aa, Artificial Sequence
  Recombinant D4
MGHHHHHHHHAFLAERLHRALKGIGTDEFTLNRIMVSRSEIDLLDIRTEFKKHYGYSLYSAIKSDTSGDYEITLLKICGGDD

SEQ ID NO: 10 - protein, 17 aa, Homo sapiens
SNAQRQLIVKEYQAAYG

SEQ ID NO: 11 - protein, 11 aa, Homo sapiens
LIVKEYQAAYG

SEQ ID NO: 12 - protein, 12 aa, Homo sapiens
IVKEYQAAYGKE

SEQ ID NO: 13 - protein, 8 aa, Homo sapiens
KEYQAAYG

SEQ ID NO: 14 - protein, 9 aa, Homo sapiens
DLSGHFEHL

SEQ ID NO: 15 - protein, 7 aa, Homo sapiens
LSGHFEH

SEQ ID NO: 16 - protein, 20 aa, Homo sapiens
ASIWVGHRGTVRDYPDFSPS

SEQ ID NO: 17 - protein, 18 aa, Homo sapiens
SIWVGHRGTVRDYPDFSP

SEQ ID NO: 18 - protein, 4 aa, Homo sapiens
YPDF

SEQ ID NO: 19 - protein, 38 aa, Homo sapiens
KEYQAAYGKELKDDLKGDLSGHFEHLMVALVTPPAVFD

SEQ ID NO: 20 - protein, 56 aa, Homo sapiens
QKAIRGIGTDEKMLISILTERSNAQRQLIVKEYQAAYGKELKDDLKGDLSGHFEHL

SEQ ID NO: 21 - protein, 26 aa, Homo sapiens
KEYQAAYGKELKDDLKGDLSGHFEHL

SEQ ID NO: 22 - protein, 17 aa, Homo sapiens
KEYQAAYGKELKDDLKG

SEQ ID NO: 23 - protein, 61 aa, Mus musculus
IRKAIRGLGTDEKTLINILTERSNAQRQLIVKQYQAAYEQELKDDLKGDLSGHFEHVMVAL

SEQ ID NO: 24 - protein, 61 aa, Rattus norvegicus
IRKAIKGIGTDEKTLINILTERSNAQRQLIVKQYQEAYEQALKADLKGDLSGHFEHVMVAL

SEQ ID NO: 25 - protein, 61 aa, Bos taurus
IRKAIRGIGTDEKTLISILTERTNAQRLLIAKEYQALCGKELKDDLKGDLSGHFKHLMVAL

SEQ ID NO: 26 - protein, 61 aa, Canis familiaris
IRKAIRGIGTEEKTLISILTERTNAQHQLIVREYQAAYGKELKDDLKGDLSGHFKQLMVAL

SEQ ID NO: 27 - protein, 61 aa, Oryctolagus cuniculus
IRKAIRGIGTDEKTLISILAERSSAQRQLIVKEYQAAYGKELKDDLKGDLSGHFEHLMVAL

SEQ ID NO: 28 - protein, 61 aa, Macaca mulatta
IRKAIRGIGTDEKMLISILTERSNAQRQLIVKEYQAAYGKELKDDLKGDLSGHFEHLMVAL

SEQ ID NO: 29 - protein, 61 aa, Gorilla gorilla; identical to SEQ ID NO: 1

SEQ ID NO: 30 - protein, 61 aa, Mus musculus
LHQALKGAGTDEFTLNRIMVSRSEIDLLDIRHEFKKHYGYSLYSAIQSDTSGDYRTVLLKI

SEQ ID NO: 31 - protein, 61 aa, Rattus norvegicus
LHQALKGAGTDEFTLNRIMVSRSEIDLLDIRREFKKHYGCSLYSAIQSDTSGDYRTVLLKI

SEQ ID NO: 32 - protein, 61 aa, Bos taurus
LYRALKGAGTDEFTLNRIMVSRSEIDLLDIRAEFKKLSGYSLYSAIKSDTSGDYEITLLKI

SEQ ID NO: 33 - protein, 61 aa, Canis familiaris
LHQALKGAGTDEFTLNRIMVSRSEIDLLDIRAEFKKHYGYSLYSAIKSDTSGDYELTLLKI

SEQ ID NO: 34 - protein, 61 aa, Oryctolagus cuniculus
LYQALKGAGTDEFTLNRIMVSRSEIDLLDIRAEFKKQYGYSLYSAIKSDTSGDYEVTLLKI

SEQ ID NO: 35 - protein, 61 aa, Macaca mulatta
LHRALKGAGTDEFTLNRIMVSRSEIDLLDIRIEFKKHYGYSLYSAIKSDTSGDYEITLLKI

SEQ ID NO: 36 - protein, 61 aa, Gorilla gorilla; identical to SEQ ID NO: 2

SEQ ID NO: 37 - protein, 17 aa, Artificial Sequence
  epitope included within the first repeat domain of mammalian Annexin A3
XXAQXXLIXXXYQXXXX

SEQ ID NO: 38 - protein, 11 aa, Artificial Sequence
  epitope included within the first repeat domain of mammalian Annexin A3
LIXXXYQXXXX

SEQ ID NO: 39 - protein, 12 aa, Artificial Sequence
  epitope included within the first repeat domain of mammalian Annexin A3
IXXXYQXXXXXX

SEQ ID NO: 40 - protein, 8 aa, Artificial Sequence
  epitope included within the first repeat domain of mammalian Annexin A3
XXYQXXXX

SEQ ID NO: 41 - protein, 17 aa, Artificial Sequence
  epitope included within the first repeat domain of mammalian Annexin A3
XXYQXXXXXXLKXDLKG

SEQ ID NO: 42 - protein, 9 aa, Artificial Sequence
  epitope included within the first repeat domain of mammalian Annexin A3
DLSGHFXXX

SEQ ID NO: 43 - protein, 7 aa, Artificial Sequence
  epitope included within the first repeat domain of mammalian Annexin A3
LSGHFXX

SEQ ID NO: 44 - protein, 17 aa, Canis familiaris
TNAQHQLIVREYQAAYG

SEQ ID NO: 45 - protein, 11 aa, Canis familiaris
LIVREYQAAYG

SEQ ID NO: 46 - protein, 12 aa, Canis familiaris
IVREYQAAYGKE

SEQ ID NO: 47 - protein, 8 aa, Canis familiaris
REYQAAYG

SEQ ID NO: 48 - protein, 9 aa, Canis familiaris
DLSGHFKQL

SEQ ID NO: 49 - protein, 7 aa, Canis familiaris
LSGHFKQ

SEQ ID NO: 50 - protein, 17 aa, Canis familiaris
REYQAAYGKELKDDLKG

SEQ ID NO: 51 - protein, 106 aa, Artificial Sequence
  vANA-7
MASIWVGHRGTVRDYPDFSPSVDAEAIQKAIRGIGTDEKMLISILTERSNAQRQLIVKEYQAAYGKELKDDLKGDLSGHFEHLMVALVTPPAVFDAKQLKKSMKGA

SEQ ID NO: 52 - protein, 72 aa, Artificial Sequence
  vANA-3
GTDEKMLISILTERSNAQRQLIVKEYQAAYGKELKDDLKGDLSGHFEHLMVALVTPPAVFDAKQLKKSMKGA

SEQ ID NO: 53 - protein, 104 aa, Artificial Sequence
  2J7
VPRGSGMKETAAAKFERQHMDSPDLHLGDTIEYQDLGTDDDDKAMAIKEYQAAYGKELKDDLKGDLSGHFEHLMVALVTPPAVFDESDPNSSSVDKLAAALEPG

SEQ ID NO: 54 - protein, 101 aa, Artificial Sequence
  2Z13
DLHLGDTIEYQDLGTDDDDKAMAIQKAIRGIGTDEKMLISILTERSNAQRQLIVKEYQAAYGKELKDDLKGDLSGHFEHLIGSEFELRRQACGRTRARVND